(19) United States
(12) Patent Application Publication
(10) Pub. No.: US 2026/0327547 A1
(43) Pub. Date: Oct. 22, 2026

(54) DEVICES AND METHODS FOR INOCULATING A TARGET

(71) Applicant: STRATIX LABS CORPORATION, St. Paul, MN (US)

(72) Inventors: Joshua ERICKSON, Blaine, MN (US); Mark MULVAHILL, St. Louis Park, MN (US); Maya BURROUGHS, St. Paul, MN (US)

(21) Appl. No.: 19/133,216

(22) PCT Filed: Nov. 29, 2023
(86) PCT No.: PCT/US2023/081566

§ 371 Date: May 27, 2025

Related U.S. Application Data
(60) Provisional application No. 63/428,886, filed on Nov. 30, 2022.

(51) Int. Cl. C12M 1/30; C12M 1/00

(52) U.S. Cl. CPC C12M 33/02; C12M 23/38

(57) ABSTRACT

Inoculating systems/devices, methods for inoculating a target, and coating methods are disclosed. An example inoculating device may include a transfer region. A cover receiving region may encompass the transfer region. A dried coating solution may be disposed within the transfer region. The dried coating solution may include viable microorganisms and one or more additives that improve survivability of the viable microorganisms. A cover may be removably secured to the cover receiving region. The cover may be adapted to protect the viable microorganisms. The inoculation device may be adapted to quickly transfer the viable microorganisms to a target from a dry state without first rehydrating the dried coating solution.

### CROSS REFERENCE TO RELATED APPLICATIONS

This application claims the benefit of U.S. Patent Application Ser. No. 63/428,886 filed on Nov. 30, 2022, the disclosure of which is incorporated herein by reference.

### TECHNICAL FIELD

The present disclosure pertains to devices and methods for inoculating a target.

### BACKGROUND

A number of devices have been developed for inoculating a target. Some of these devices include microbe transfer devices and/or the like. Of the known devices and methods, each has certain advantages and disadvantages. There is an ongoing need to provide alternative devices as well as alternative methods for manufacturing and using devices, for example devices for inoculating a target.

### BRIEF SUMMARY

This disclosure provides design, material, manufacturing method, and use alternatives for inoculating devices. An example may be found in an inoculation device. The inoculation device includes a transfer region, a cover receiving region encompassing the transfer region, and a dried coating solution disposed within the transfer region, the dried coating solution including viable microorganisms and one or more additives that improve survivability of the viable microorganisms. A cover is removably secured to the cover receiving region, the cover adapted to protect the viable microorganisms. The inoculation device is adapted to quickly transfer the viable microorganisms to a target from a dry state without first rehydrating the dried coating solution.

Alternatively or additionally, the viable microorganisms may be present in the dried coating solution in a quantity between a desired lower limit of colony forming units (CFUs) and a desired upper limit of CFUs.

Alternatively or additionally, the quantity of viable microorganisms present in the dried coating solution may maintain between the desired lower limit and desired upper limit of colony forming units for at least 90 days.

Alternatively or additionally, at least 90 percent of the viable microorganisms within the dried coating solution may be substantially transferred to the target in 60 seconds or less at a temperature between about 15° C. to about 25° C.

Alternatively or additionally, the quantity of viable microorganisms within the dried coating solution may be between about 500 CFUs and about 12,500 CFUs.

Alternatively or additionally, the quantity of viable microorganisms within the dried coating solution may be between about 10⁴ CFUs and about 10⁹ CFUs.

Alternatively or additionally, the inoculation device may be disposed within a moisture-impermeable container.

Alternatively or additionally, the moisture-impermeable container may include a desiccant disposed within the moisture-impermeable container.

Alternatively or additionally, the moisture-impermeable container may include an oxygen scavenger disposed within the moisture-impermeable container.

Alternatively or additionally, the moisture-impermeable container may include an inert gas disposed within the moisture-impermeable container.

Another example may be found in an inoculating system. The inoculating system includes an inoculating member having a transfer region and a cover receiving region, the inoculating member adapted for storing and transferring viable microorganisms to a target. A coating solution is dried on the transfer region, the coating solution including viable microorganisms and one or more additives that improve survivability of the viable microorganisms. A cover is removably secured to the cover receiving region and disposed over the transfer region, the cover adapted to protect the viable microorganisms. The inoculating member and cover are further sealed in a moisture barrier container defining a packaging environment within the moisture barrier container. The viable microorganisms dried on the transfer region stabilize within a desired lower limit and a desired upper limit of colony forming units and maintain within the desired lower limit and desired upper limit of colony forming units for at least 90 days. The viable microorganisms define an inoculum. The inoculating member is configured to transfer the viable microorganisms to a target from the inoculating member in a dry state without the need for an additional step to prepare the inoculum. The viable microorganisms are rapidly transferred to the target.

Alternatively or additionally, the target may include a solid media, a liquid solution, or a microbial collection device.

Alternatively or additionally, the viable microorganisms may be substantially transferred to the target in 60 seconds or less.

Alternatively or additionally, the viable microorganisms may be substantially transferred to the target in 30 seconds or less.

Alternatively or additionally, the viable microorganisms may be substantially transferred to the target in 10 seconds or less.

Alternatively or additionally, the viable microorganisms may be substantially transferred to the target in 60 seconds or less at a temperature between about 15° C. to about 25° C.

Alternatively or additionally, the viable microorganisms may be substantially transferred to the target in 30 seconds or less at a temperature between about 15° C. to about 25° C.

Alternatively or additionally, the viable microorganisms may be substantially transferred to the target in 10 seconds or less at a temperature between about 15° C. to about 25° C.

Alternatively or additionally, at least 90% of the viable microorganisms may be transferred to the target in 60 seconds or less at a temperature between about 15° C. to about 25° C.

Alternatively or additionally, at least 90% of the viable microorganisms may be transferred to the target in 30 seconds or less at a temperature between about 15° C. to about 25° C.

Alternatively or additionally, at least 90% of the viable microorganisms may be transferred to the target in 10 seconds or less at a temperature between about 15° C. to about 25° C.

Alternatively or additionally, the quantity of viable microorganisms stabilized on the transfer region may be between about 10 colony forming units and 15,000 colony forming units.

Alternatively or additionally, the quantity of viable microorganisms stabilized on the transfer region may be between about 500 colony forming units and 12,500 colony forming units.

Alternatively or additionally, the quantity of viable microorganisms stabilized on the transfer region may be between about 500 colony forming units and 2,500 colony forming units.

Alternatively or additionally, the quantity of viable microorganisms stabilized on the transfer region may be between about 500 colony forming units and 1,500 colony forming units.

Alternatively or additionally, the quantity of viable microorganisms stabilized on the transfer may be is between about 10⁴ colony forming units and 10⁹ colony forming units.

Alternatively or additionally, the quantity of viable microorganisms stabilized on the transfer region may be between about 10⁴ colony forming units and 10⁸ colony forming units.

Alternatively or additionally, the quantity of viable microorganisms stabilized on the transfer region may be between about 10⁴ colony forming units and 10⁷ colony forming units.

Alternatively or additionally, the quantity of viable microorganisms stabilized on the transfer region may be between about 10⁴ colony forming units and 10⁶ colony forming units.

Alternatively or additionally, the quantity of viable microorganisms maintains within the desired lower limit and desired upper limit of colony forming units for at least 180 days.

Alternatively or additionally, the quantity of viable microorganisms maintains within the desired lower limit and desired upper limit of colony forming units for at least 365 days.

Alternatively or additionally, the quantity of viable microorganisms maintains within the desired lower limit and desired upper limit of colony forming units for at least 730 days.

Alternatively or additionally, the inoculating system may be manufactured as part of a batch and the quantity of viable microorganisms stabilized on the transfer region of the inoculating system may be between about 50% to about 200% of the batch's mean quantity of viable microorganisms per device.

Alternatively or additionally, a quantity of between about 10 colony forming units and 15,000 colony forming units may be transferred to the target.

Alternatively or additionally, a quantity of between about 500 colony forming units and 12,500 colony forming units may be transferred to the target.

Alternatively or additionally, a quantity of between about 500 colony forming units and 2,500 colony forming units may be transferred to the target.

Alternatively or additionally, a quantity of between about 500 colony forming units and 1,500 colony forming units may be transferred to the target.

Alternatively or additionally, a quantity of between about 10⁴ colony forming units and 10⁹ colony forming units may be transferred to the target.

Alternatively or additionally, a quantity of between about 10⁴ colony forming units and 10⁸ colony forming units may be transferred to the target.

Alternatively or additionally, a quantity of between about 10⁴ colony forming units and 10⁷ colony forming units may be transferred to the target.

Alternatively or additionally, a quantity of between about 10⁴ colony forming units and 10⁶ colony forming units may be transferred to the target.

Alternatively or additionally, the moisture barrier container may include a modified atmosphere.

Alternatively or additionally, the moisture barrier container may include a desiccant.

Alternatively or additionally, the moisture barrier container may include an oxygen scavenger.

Alternatively or additionally, the moisture barrier container may include an inert gas.

Alternatively or additionally, the inoculating system may include a mechanism to enable airflow between the transfer region and the packaging environment of the moisture barrier container.

Alternatively or additionally, the inoculating system may include a vent or opening to enable airflow between the transfer region and the packaging environment of the moisture barrier container.

Alternatively or additionally, the cover or the cover receiving region may include a mechanism to enable airflow between the transfer region and the packaging environment of the moisture barrier container.

Alternatively or additionally, the cover may include a vent or opening to enable airflow between the transfer region and the packaging environment of the moisture barrier container.

Alternatively or additionally, a desiccant or an oxygen scavenger may be ontained within the cover.

Alternatively or additionally, the moisture barrier container may be further sealed within a secondary container.

Another example may be found in an inoculation device. The inoculation device includes an elongate polymeric body including a cover region encompassing a distal portion of the elongate polymeric body, a transfer region disposed within the cover region of the elongated polymeric body, and a handle region encompassing a proximal portion of the elongate polymeric body. A dried coating solution is disposed within the transfer region, the dried coating solution including viable microorganisms and one or more additives that improve survivability of the viable microorganisms. A cover is removably secured relative to the coverable region, the cover adapted to protect the viable microorganisms. The inoculation device is adapted to quickly transfer the viable microorganisms to a target from a dry state without first rehydrating the dried coating solution.

Alternatively or additionally, the inoculation device may further include a cover securing feature disposed within a proximal portion of the cover region.

Alternatively or additionally, the cover securing feature may be adapted to form an interference fit with the cover.

Alternatively or additionally, the handle region may include a grip region that is adapted to improve grasping of the handle region.

Alternatively or additionally, the elongate polymeric body may include an elongate injection molded polymeric body.

Alternatively or additionally, the elongate polymeric body may have an overall length of about 10 cm to about 30 cm.

Alternatively or additionally, the cover region may have a length in a range of about 10 percent to about 60 percent of the overall length of the elongate polymeric body.

Alternatively or additionally, the handle region may have a length in a range of about 40 percent to about 90 percent of the overall length of the elongate polymeric body.

Alternatively or additionally, the elongate polymeric body may have a diameter of about 0.5 cm to about 1.5 cm.

Alternatively or additionally, the viable microorganisms may be present in the dried coating solution in a quantity between a desired lower limit of colony forming units (CFUs) and a desired upper limit of CFUs.

Alternatively or additionally, the quantity of viable microorganisms present in the dried coating solution maintains between the desired lower limit and desired upper limit of colony forming units for at least 90 days.

Alternatively or additionally, at least 90 percent of the viable microorganisms within the dried coating solution may be substantially transferred to the target in 60 seconds or less at a temperature between about 15° C. to about 25° C.

Alternatively or additionally, the quantity of viable microorganisms within the dried coating solution may be between about 500 CFUs and about 12,500 CFUs.

Alternatively or additionally, the quantity of viable microorganisms within the dried coating solution may be between about 10⁴ CFUs and about 10⁹ CFUs.

Alternatively or additionally, the inoculation device may be disposed within a moisture-impermeable container.

Alternatively or additionally, the moisture-impermeable container may include a desiccant disposed within the moisture-impermeable container.

Alternatively or additionally, the moisture-impermeable container may include an oxygen scavenger disposed within the moisture-impermeable container.

Alternatively or additionally, the moisture-impermeable container may include an inert gas disposed within the moisture-impermeable container.

Alternatively or additionally, the transfer region may include a distal end surface of the elongate polymeric body.

Another example may be found in an inoculating system. The inoculating system includes a disposable inoculation device adapted for storing and rapidly transferring viable microorganisms to a target. The disposable inoculation device has an elongate body that includes a handle region having a textured surface disposed along at least a portion of the handle region, the textured surface adapted to aid in gripping and manipulating the disposable inoculation device. The elongate body includes a transfer region for storing and transferring viable microorganisms, said transfer region positioned at an extremity of the elongate body, the transfer region distal of the handle region. The elongate body includes a cap receiving region encompassing the transfer region, the cap receiving region including a securement feature adapted to releasably secure a cap. A coating solution is dried on the transfer region and includes viable microorganisms and one or more additives that improve survivability of the viable microorganisms. A cap is removably disposed over the cap receiving region without contacting the transfer region, a modified atmosphere disposed within the cap and thus the transfer region is exposed to the modified atmosphere. The inoculation device is in a sealed moisture barrier container. The viable microorganisms define an inoculum. The inoculating member is configured to transfer the viable microorganisms to a target from the inoculating member in a dry state without the need for an additional step to prepare the inoculum. The viable microorganisms are substantially transferred to the target in 60 seconds or less.

Alternatively or additionally, the target may be a solid media, a liquid, or a microbial collection device.

Alternatively or additionally, the target may be an agar media and the inoculating member may be configured to isolate a colony forming unit on the agar media by performing a direct streak plate method without the need for an additional instrument to spread the cells.

Alternatively or additionally, the target may be a liquid contained within a vessel.

Alternatively or additionally, the inoculating system may be configured to insert the transfer region into a vessel without introducing contamination.

Alternatively or additionally, the inoculating system may be configured to insert the transfer region at least 1 cm into a vessel.

Alternatively or additionally, the viable microorganisms may be substantially transferred to the target in 10 seconds or less.

Alternatively or additionally, the viable microorganisms may be substantially transferred to the target in 10 seconds or less at a temperature between about 15° C. to about 25° C.

Alternatively or additionally, at least 90% of the viable microorganisms may be transferred to the target in 10 seconds or less at a temperature between about 15° C. to about 25° C.

Alternatively or additionally, the quantity of viable microorganisms stabilized on the transfer region may be between about 500 colony forming units and 1,500 colony forming units.

Alternatively or additionally, the quantity of viable microorganisms stabilized on the transfer region may be between about 10⁴ colony forming units and 10⁸ colony forming units.

Alternatively or additionally, the quantity of viable microorganisms maintains within the desired lower limit and desired upper limit of colony forming units for at least 365 days.

Alternatively or additionally, the inoculating system may be manufactured as part of a batch and the quantity of viable microorganisms stabilized on the transfer region of the inoculating system may be between about 50% to about 200% of the batch's mean quantity of viable microorganisms per device.

Alternatively or additionally, a quantity of between about 500 colony forming units and 1,500 colony forming units may be transferred to the target.

Alternatively or additionally, a quantity of between about 10⁴ colony forming units and 10⁸ colony forming units may be transferred to the target.

Alternatively or additionally, the moisture barrier container may include a modified atmosphere.

Another example may be found in an inoculation device. The inoculation device includes a disposable vessel comprising a vessel wall having an outside wall surface and an inside wall surface defining an interior volume and a transfer region disposed on a portion of the inside wall surface. A dried coating solution is disposed within the transfer region, the dried coating solution including viable microorganisms and one or more additives that improve survivability of the viable microorganisms. A cover is removably securable to the disposable vessel, the cover adapted to protect the viable microorganisms. The inoculation device is adapted to quickly transfer the viable microorganisms from the transfer region to a liquid target transferred into the disposable vessel.

Alternatively or additionally, the disposable vessel may include an open end and a closed end, and the open end may be adapted to releasably secure the cover thereto.

Alternatively or additionally, the disposable vessel may include a test tube.

Alternatively or additionally, the viable microorganisms may be present in the dried coating solution in a quantity between a desired lower limit of colony forming units (CFUs) and a desired upper limit of CFUs.

Alternatively or additionally, the quantity of viable microorganisms present in the dried coating solution maintains between the desired lower limit and desired upper limit of colony forming units for at least 90 days.

Alternatively or additionally, at least 90 percent of the viable microorganisms within the dried coating solution may be substantially transferred to the liquid target in 60 seconds or less at a temperature between about 15° C. to about 25° C.

Alternatively or additionally, the quantity of viable microorganisms within the dried coating solution may be between about 500 CFUs and about 12,500 CFUs.

Alternatively or additionally, the quantity of viable microorganisms within the dried coating solution may be between about 10⁴ CFUs and about 10⁹ CFUs.

Alternatively or additionally, the inoculation device may be disposed within a moisture-impermeable container.

Alternatively or additionally, the moisture-impermeable container may include a desiccant disposed within the moisture-impermeable container.

Alternatively or additionally, the moisture-impermeable container may include an oxygen scavenger disposed within the moisture-impermeable container.

Alternatively or additionally, the moisture-impermeable container may include an inert gas disposed within the moisture-impermeable container.

Another example may be found in an inoculating system. The inoculating system includes a disposable inoculating vessel including a vessel interior adapted to receive a liquid, a vessel exterior adapted to enable grip and manipulation of the vessel, a transfer region disposed within the vessel interior, and a coating solution dried onto the transfer region, the coating solution comprising viable microorganisms and one or more additives that improve survivability of the viable microorganisms. A cap is removably securable to the disposable inoculating vessel, the cap adapted to protect the viable microorganisms. The inoculating member and cap are further sealed in a moisture barrier container. The viable microorganisms dried on the transfer region stabilize within a desired lower limit and a desired upper limit of colony forming units. The quantity of viable microorganisms maintains within the desired lower limit and desired upper limit of colony forming units for at least 90 days. The viable microorganisms define an inoculum. The inoculating member is configured to transfer the viable microorganisms to a liquid target from the transfer region in a dry state without the need for an additional step to prepare the inoculum. The viable microorganisms are substantially transferred to the liquid target in 60 seconds or less.

Alternatively or additionally, the vessel may be configured to receive and store a volume of at least 0.25 mL.

Alternatively or additionally, the vessel may be configured to receive and store a volume of at least 1 mL.

Alternatively or additionally, the viable microorganisms may be substantially transferred to the liquid target in 10 seconds or less.

Alternatively or additionally, the viable microorganisms may be substantially transferred to the target in 10 seconds or less at a temperature between about 15° C. to about 25° C.

Alternatively or additionally, at least 90% of the viable microorganisms may be transferred to the target in 10 seconds or less at a temperature between about 15° C. to about 25° C.

Alternatively or additionally, the quantity of viable microorganisms stabilized on the transfer region may be between about 500 colony forming units and 1,500 colony forming units.

Alternatively or additionally, the quantity of viable microorganisms stabilized on the transfer region may be between about 10⁴ colony forming units and 10⁸ colony forming units.

Alternatively or additionally, the quantity of viable microorganisms maintains within the desired lower limit and desired upper limit of colony forming units for at least 365 days.

Alternatively or additionally, the inoculating system may be manufactured as part of a batch and the quantity of viable microorganisms stabilized on the transfer region of the inoculating system may be between about 50% to about 200% of the batch's mean quantity of viable microorganisms per device.

Alternatively or additionally, a quantity of between about 500 colony forming units and 1,500 colony forming units may be transferred to the liquid target.

Alternatively or additionally, a quantity of between about 10⁴ colony forming units and 10⁸ colony forming units may be transferred to the liquid target.

Alternatively or additionally, the moisture barrier container may include a modified atmosphere.

Another example may be found in an inoculating system. The inoculating system includes a disposable inoculating member including a transfer region and a cover receiving region, wherein the inoculating member is adapted to store and transfer viable microorganisms to a microbial collection device, wherein the transfer region includes a planar surface configured to receive a microbial collection device. A coating solution is dried on the transfer region, the coating solution including viable microorganisms and one or more additives that improve survivability of the viable microorganisms. A cover is removably secured to the cover receiving region and disposed over the transfer region, the cover adapted to protect the viable microorganisms. The inoculating member and cover are further sealed in a moisture barrier container. The viable microorganisms dried on the transfer region stabilize within a desired lower limit and a desired upper limit of colony forming units. The quantity of viable microorganisms maintains within the desired lower limit and desired upper limit of colony forming units for at least 90 days. The viable microorganisms define an inoculum. The inoculating member is configured to transfer the viable microorganisms to a microbial collection device from the inoculating member in a dry state without the need for an additional step to prepare the inoculum. The viable microorganisms are substantially transferred to the microbial collection device by applying the microbial collection device to the transfer region.

Alternatively or additionally, the inoculating member may be adapted to be secured in a fixed position during application of a microbial collection device.

Alternatively or additionally, the inoculating system may include structure for securing the inoculating member in place during application of a microbial collection device.

Alternatively or additionally, the inoculating system may include a mechanism external to the inoculating member that is configured to secure the inoculating member in a fixed position during application of a microbial collection device.

Alternatively or additionally, the inoculating member may include a handle region along at least a portion of the inoculating member exterior to aid in gripping and manipulating the inoculating member.

Alternatively or additionally, the transfer region may have a surface area of at least 25 cm².

Alternatively or additionally, the transfer region may include a smooth surface.

Alternatively or additionally, the transfer region may include a rough surface.

Alternatively or additionally, the transfer region may include a bumpy surface.

Alternatively or additionally, the coating solution may be deposited dried on the transfer region as a plurality of drops.

Alternatively or additionally, the target may be a swab microbial collection device.

Alternatively or additionally, the target may be a sponge microbial collection device.

Alternatively or additionally, the target may be a contact plate.

Alternatively or additionally, the viable microorganisms may be readily transferred from the transfer region upon contact with the microbial collection device.

Alternatively or additionally, the quantity of viable microorganisms stabilized on the transfer region may be between about 50 colony forming units and 15,000 colony forming units.

Alternatively or additionally, the quantity of viable microorganisms stabilized on the transfer region may be between about 50 colony forming units and 500 colony forming units.

Alternatively or additionally, the quantity of viable microorganisms stabilized on the transfer region may be between about 500 colony forming units and 2,500 colony forming units.

Alternatively or additionally, the quantity of viable microorganisms stabilized on the transfer region may be between about 7,500 colony forming units and 12,500 colony forming units.

Alternatively or additionally, the quantity of viable microorganisms maintains within the desired lower limit and desired upper limit of colony forming units for at least 365 days.

Alternatively or additionally, the inoculating system may be manufactured as part of a batch and the quantity of viable microorganisms stabilized on the transfer region of the inoculating system may be between about 50% to about 200% of the batch's mean quantity of viable microorganisms per device.

Alternatively or additionally, a quantity of between about 50 colony forming units and 15,000 colony forming units may be transferred to the target.

Alternatively or additionally, a quantity of between about 50 colony forming units and 500 colony forming units may be transferred to the target.

Alternatively or additionally, a quantity of between about 500 colony forming units and 2,500 colony forming units may be transferred to the target.

Alternatively or additionally, a quantity of between about 2,500 colony forming units and 7,500 colony forming units may be transferred to the target.

Alternatively or additionally, a quantity of between about 7,500 colony forming units and 15,000 colony forming units may be transferred to the target.

Alternatively or additionally, the moisture barrier container may include a modified atmosphere.

Another example may be found in a method of storing viable microorganisms and rapidly transferring the viable microorganisms to a target. The method includes storing a moisture barrier container that an inoculating system therein, the inoculating system including an inoculating member including a transfer region and a cover receiving region, wherein the inoculating member is adapted to store and transfer viable microorganisms to a target, a coating solution that is dried on the transfer region, the coating solution including viable microorganisms and one or more additives that improve survivability of the viable microorganisms, and a cover useful for protecting the viable microorganisms removably secured to the cover receiving region and disposed over the transfer region. The inoculating member and cover are sealed in the moisture barrier container. The viable microorganisms dried on the transfer region stabilize within a desired lower limit and a desired upper limit of colony forming units. The viable microorganisms define an inoculum. The method includes removing the inoculating system from the moisture barrier container, removing the cover from the transfer region of the inoculating system, and contacting the transfer region with a biologically acceptable target. The inoculating member transfers the viable microorganisms to the target from the inoculating member in a dry state without the need for an additional step to prepare the inoculum. The viable microorganisms are substantially transferred to the target in 60 seconds or less.

Alternatively or additionally, the target may be a solid media, a liquid solution, or a microbial collection device.

Alternatively or additionally, the viable microorganisms may be substantially transferred to the target in 30 seconds or less.

Alternatively or additionally, the viable microorganisms may be substantially transferred to the target in 10 seconds or less.

Alternatively or additionally, the viable microorganisms may be substantially transferred to the target in 60 seconds or less at a temperature between about 15° C. to about 25° C.

Alternatively or additionally, the viable microorganisms may be substantially transferred to the target in 30 seconds or less at a temperature between about 15° C. to about 25° C.

Alternatively or additionally, the viable microorganisms may be substantially transferred to the target in 10 seconds or less at a temperature between about 15° C. to about 25° C.

Alternatively or additionally, at least 90% of the viable microorganisms may be transferred to the target in 60 seconds or less at a temperature between about 15° C. to about 25° C.

Alternatively or additionally, at least 90% of the viable microorganisms may be transferred to the target in 30 seconds or less at a temperature between about 15° C. to about 25° C.

Alternatively or additionally, at least 90% of the viable microorganisms may be transferred to the target in 10 seconds or less at a temperature between about 15° C. to about 25° C.

Alternatively or additionally, the quantity of viable microorganisms stabilized on the transfer region may be between about 10 colony forming units and 15,000 colony forming units.

Alternatively or additionally, the quantity of viable microorganisms stabilized on the transfer region may be between about 500 colony forming units and 12,500 colony forming units.

Alternatively or additionally, the quantity of viable microorganisms stabilized on the transfer region may be between about 500 colony forming units and 2,500 colony forming units.

Alternatively or additionally, the quantity of viable microorganisms stabilized on the transfer region may be between about 500 colony forming units and 1,500 colony forming units.

Alternatively or additionally, the quantity of viable microorganisms stabilized on the transfer region may be between about 10⁴ colony forming units and 10⁹ colony forming units.

Alternatively or additionally, the quantity of viable microorganisms stabilized on the transfer region may be between about 10⁴ colony forming units and 10⁸ colony forming units.

Alternatively or additionally, the quantity of viable microorganisms stabilized on the transfer region may be between about 10⁴ colony forming units and 10⁷ colony forming units.

Alternatively or additionally, the quantity of viable microorganisms stabilized on the transfer region may be between about 10⁴ colony forming units and 10⁶ colony forming units.

Alternatively or additionally, the quantity of viable microorganisms maintains within the desired lower limit and desired upper limit of colony forming units for at least 90 days.

Alternatively or additionally, the quantity of viable microorganisms maintains within the desired lower limit and desired upper limit of colony forming units for at least 180 days.

Alternatively or additionally, the quantity of viable microorganisms maintains within the desired lower limit and desired upper limit of colony forming units for at least 365 days.

Alternatively or additionally, the quantity of viable microorganisms maintains within the desired lower limit and desired upper limit of colony forming units for at least 730 days.

Alternatively or additionally, the inoculating system may be manufactured as part of a batch and the quantity of viable microorganisms stabilized on the transfer region of the inoculating system may be between about 50% to about 200% of the batch's mean quantity of viable microorganisms per device.

Alternatively or additionally, a quantity of between about 10 colony forming units and 15,000 colony forming units may be transferred to the target.

Alternatively or additionally, a quantity of between about 500 colony forming units and 12,500 colony forming units may be transferred to the target.

Alternatively or additionally, a quantity of between about 500 colony forming units and 2,500 colony forming units may be transferred to the target.

Alternatively or additionally, a quantity of between about 500 colony forming units and 1,500 colony forming units may be transferred to the target.

Alternatively or additionally, a quantity of between about 10⁴ colony forming units and 10⁹ colony forming may be are transferred to the target.

Alternatively or additionally, a quantity of between about 10⁴ colony forming units and 10⁸ colony forming units may be transferred to the target.

Alternatively or additionally, a quantity of between about 10⁴ colony forming units and 10⁷ colony forming units may be transferred to the target.

Alternatively or additionally, a quantity of between about 10⁴ colony forming units and 10⁶ colony forming units may be transferred to the target.

Alternatively or additionally, the moisture barrier container may include a modified atmosphere.

Alternatively or additionally, the moisture barrier container may include a desiccant.

Alternatively or additionally, the moisture barrier container may include an oxygen scavenger.

Alternatively or additionally, the moisture barrier container may include an inert gas.

Alternatively or additionally, the inoculating system may include a mechanism to enable airflow between the transfer region and the packaging environment of the moisture barrier container.

Alternatively or additionally, the inoculating system may include a vent or opening to enable airflow between the transfer region and the packaging environment of the moisture barrier container.

Alternatively or additionally, the cover or the cover receiving region may include a mechanism to enable airflow between the transfer region and the packaging environment of the moisture barrier container.

Alternatively or additionally, the cover may include a vent or opening to enable airflow between the transfer region and the packaging environment of the moisture barrier container.

Alternatively or additionally, a desiccant or an oxygen scavenger may be contained within the cover.

Alternatively or additionally, the moisture barrier container may be further sealed within a secondary container.

Alternatively or additionally, the inoculating system may be sealed and stored in the moisture barrier container for at least about 30 days.

Alternatively or additionally, the inoculating system may be sealed and stored in the moisture barrier container for at least about 60 days.

Alternatively or additionally, the inoculating system may be sealed and stored in the moisture barrier container for at least about 90 days.

Alternatively or additionally, the inoculating system may be sealed and stored in the moisture barrier container for at least about 180 days.

Alternatively or additionally, the inoculating system may be sealed and stored in the moisture barrier container for at least about 365 days.

Alternatively or additionally, the target may be a solid growth media and the inoculating member may be used to distribute the viable microorganisms on the target.

Alternatively or additionally, the target may be a solid growth media and the inoculating member may transfer the viable microorganisms to the target and may generate at least one isolated colony forming unit.

Alternatively or additionally, the target may be a solid growth media and the inoculating member may transfer the viable microorganisms to the target and may generate at least one isolated colony forming unit without the need for an additional instrument to distribute the viable microorganisms on the target.

Alternatively or additionally, the transfer region may be located at the end of an elongated rod shape and the transfer region may be inserted into a vessel comprising a liquid solution target.

Alternatively or additionally, the transfer region may be located at the end of an elongated rod shape and an extension region may connect the transfer region to the cover receiving region, and the transfer region and extension region may be inserted into a vessel that includes a liquid solution target.

Alternatively or additionally, the target may be a liquid solution and the inoculating member may include a vessel useful for receiving and storing a liquid, and the liquid target may be dispensed into the vessel of the inoculating member and may submerge the transfer region.

Alternatively or additionally, the target may be a microbial collection device and the microbial collection device may be applied to the transfer region.

Alternatively or additionally, the inoculating member may be secured in place during transfer of the viable microorganisms to the target.

Alternatively or additionally, the inoculating member may be secured in place by an operator gripping and manipulating the inoculating member during transfer of the viable microorganisms to the target.

Alternatively or additionally, the inoculating member may be secured in place by an external instrument during transfer of the viable microorganisms to the target.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of an illustrative inoculating device.

FIG. 2 is a perspective view of an illustrative inoculating device.

FIG. 3 is a top view of an illustrative inoculating device.

FIGS. 4-5 depict an illustrative inoculating device and an example cap.

FIG. 6 illustrates an illustrative inoculating device disposed within a package.

FIG. 7 is a perspective view of an illustrative inoculating device.

FIG. 8 is a perspective view of an illustrative inoculating device.

FIG. 9 is a perspective view of an illustrative inoculating device.

FIG. 10 is a perspective view of an illustrative inoculating device.

FIG. 11 is a side view of the illustrative inoculating device of FIG. 10, shown with a cover and disposed within a moisture barrier container.

FIG. 12 is a side view of an illustrative inoculating device.

FIG. 13 is a cross-sectional view taken along the line 13-13 of FIG. 12.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Precise and controlled inoculation and/or transfer of microorganisms to a target is useful in a number of different settings. For example, in a number of laboratory settings it may be desirable for control and/or validation testing that demonstrates the viability of certain targets. In some instances, the target may include an agar plate. Example testing processes may include growth promotion testing, environmental testing, and/or the like. In other instances, it may be desirable to transfer or utilize a precise number of microorganisms. Disclosed herein are devices and methods for inoculating and/or transferring microorganisms to a target.

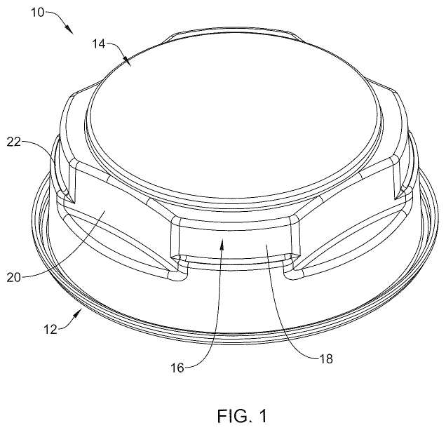

FIG. 1 is a perspective view of an illustrative inoculation device 10. The device 10 may have a number of uses such as for inoculating a target/substrate. The target (not shown in FIG. 1) may be a solid target (e.g., an agar plate) or a liquid target or media. The device 10 may include a base or handle region 12 and a transfer region 14. The handle region 12 may be configured so that a user can grip or hold the device 10 during use. The transfer region 14 may have a pre-determined quantity of viable microorganisms disposed thereon. The microorganisms may be disposed/coated onto the transfer region 14 and dried/dehydrated in a manner that maintains the viability of the microorganism and that allows for transfer of the microorganisms in a relatively simple manner that does not require rehydration of the microorganisms prior to an inoculating/transfer process (e.g., prior to contacting the target). Thus, the use of the device 10 does not require any additional rehydration steps (e.g., where the microorganisms are rehydrated) in order to efficiently transfer the microorganisms to the target in a relatively simple, straightforward manner such as merely contacting the transfer region 14 with the target.

While in some instances it may be desirable to utilize a pre-determined quantity of microorganisms, in other instances knowing the quantity of microorganisms may not be required. Therefore, in the examples disclosed herein, either a pre-determined quantity of microorganisms may be utilized or a quantity of microorganisms that is not precisely known may be utilized.

The transfer region 14 may have a suitable shape, size, and material composition that aids in facilitating the transfer of microorganisms disposed thereon to a target. For example, the transfer region 14 may have a substantially round or rounded shape as depicted in FIG. 1. Other shapes such as regular shapes, irregular shapes, and/or polygonal shapes including regular polygons and irregular polygons (e.g., having 1, 2, 3, 4, 5, 6, 7, 8, 9, 10, or more sides) may be utilized for the transfer region 14. In some instances, the transfer region 14 may be convex or otherwise bow/arc away from the handle region 12. In other instances, the transfer region 14 may be substantially planar. In at least some instances, the sides or peripheral surfaces of the transfer region 14 may be rounded, blunt, unsharp, or the like.

In some instances, the transfer region 14 may be formed from or otherwise include a suitable material such as metal, steel, glass, plastic/polymer, and/or the like. Some examples of suitable polymers may include polypropylene, polyethylene, polyethylene terephthalate, glycol modified polyethylene terephthalate, polystyrene, poly-lactic acid, acrylonitrile butadiene styrene, polyetheretherketone, polyoxymethylene, nylon, polycarbonate, polytetrafluoroethylene, combinations thereof, and/or the like. In some of these and in other instances, the transfer region 14 may be formed from a non-water soluble material. In some of these and in other instances, the transfer region 14 may be formed from a substantially non-porous material and/or otherwise be considered to be non-porous. The device 10 may be manufactured using a suitable process such as a thermoforming, injection molding, molding, casting, and/or the like.

The transfer region 14 (e.g., when having a round or rounded shape) may have a size or diameter of about 10 mm or more, or about 20 mm or more, or about 30 mm or more, or about 10-200 mm. The surface area of the transfer region 14 may be about 15,000 mm² or less, or about 10,000 mm² or less, or about 9,000 mm² or less, or about 5,000 mm² or less, or about 10 mm² to 10,000 mm², or about 0.75 mm² or more, or about 3 mm² or more, or at least 200 mm² (e.g., at least 250 mm², at least 300 mm², at least 350 mm², at least 400 mm², at least 450 mm², at least 500 mm², at least 550 mm², at least 600 mm², at least 650 mm², at least 700 mm², at least 750 mm², at least 800 mm², at least 850 mm², at least 900 mm², at least 950 mm², or at least 1,000 mm²). The dimensions of the transfer region 14 may be selected in order to accommodate a particular target (e.g., the transfer region 14 may be sized for use with an agar plate). In addition, the size/dimensions may be selected so that the transfer region 14 can be brought into contact with target and allow for spreading action with on the target. For example, in some instances the target may have a surface area of at least 200 mm² (e.g., at least 250 mm², at least 300 mm², at least 350 mm², at least 400 mm², at least 450 mm², at least 500 mm², at least 550 mm², at least 600 mm², at least 650 mm², at least 700 mm², at least 750 mm², at least 800 mm², at least 850 mm², at least 900 mm², at least 950 mm², or at least 1,000 mm²).

The device 10 may also include a cap receiving region 16. The cap receiving region 16 may include a generally cylindrical section 18. One or more spacer sections 20 may be defined about the cylindrical section 18. The spacer sections 20 may be disposed or generally be oriented radially inward of the cylindrical section 18. In some instances, the spacer sections 20 may be or resemble cutouts in the cylindrical section 18. The orientation of the spacer sections 20 may allow for some amount of air flow or venting along the cap receiving region 16. The cap receiving region 16 may also include a recessed or grooved region 22 that may be configured to receive a cap as described in more detail herein.

In use, the device 10 may be brought into contact with the target to transfer the microorganisms. When the device 10 is disposed within a package, the process may include opening the package and removing the device 10 from the package. When the target is a solid target, this may include bringing the transfer region 14 into contact with the target. In some instances, bringing the transfer region 14 into contact with the target may include pressing the transfer region 14 onto/against/along the target, stamping the transfer region 14 onto/against/along the target, brushing the transfer region 14 onto/against/along the target, spreading the transfer region 14 onto/against/along the target, imprinting the transfer region 14 onto the target, contacting the transfer region 14 with the target and then spreading the pre-determined quantity of viable microorganisms along the target, combinations thereof, and/or the like. In some instances, after being brought into contact with the target to transfer the microorganisms, the transfer region 14 may be configured to remain in contact with the target for a contact time (e.g., about 30 seconds or less, or about 15 seconds or less, or about 10 seconds or less, or about 5 seconds or less, or about 1 second or less). In some of these and in other instances, the transfer region 14 may be configured to remain in contact indefinitely. In some instances, a spreading action may be utilized after contacting the transfer region 14 with the target. In other instances, a spreading action is not required (e.g., the transfer region 14 is configured to transfer the pre-determined quantity of viable microorganisms to a target during an inoculation operation without having to conduct a spreading operation). If desired, the transfer region 14 may be removed from contact with the target.

The target may be incubated for a suitable incubation time. After the suitable incubation time, a user may observe the target for an indication of growth. Observing the target for an indication of growth may include a qualitative assessment, a quantitative assessment, evaluating viability, evaluating fluorescence, detection of a fluorescent microorganism, evaluating pH, evaluating a change in color, enumeration of colony forming units, manual colony counting, automated colony counting, enumeration of 1-100 colony forming units, visually observing a predetermined pattern on the culture medium, combinations thereof, and/or the like.

When the transfer region 14 is brought into contact with the target, the microorganisms may essentially simultaneously become at least partially rehydrated due to the water content of the target itself and transfer the microorganisms to the target. For example, the target may have a water content of about 5% or more, or about 10% or more. Because the microorganisms are able to become sufficiently hydrated/transferred by simply contacting the transfer region 14 with the target, a separate hydrating step/process where the microorganisms are hydrated prior to contact with the target or prior to the inoculating/transfer process is not required.

When the target is a liquid (e.g., a liquid broth, a liquid media, a liquid buffer, a saline solution, water, combinations thereof, and the like), the transfer region 14 may be submerged within the liquid. In some instances, the transfer region 14 may be swirled or agitated to facilitate simultaneous hydration and transfer upon contact between the transfer region 14 and the liquid. Again, because the microorganisms are able to become sufficiently hydrated/transferred by simply contacting the transfer region 14 with the liquid, a separate hydrating step/process where the microorganisms are hydrated prior to contact with the target and/or prior to the inoculating/transfer process is not required. In some instances, the device 10 may be tailored for transferring microorganisms to liquid. For example, the device 10 may take the form of a rod having a transfer region (e.g., similar to the transfer region 14) and a handle region (e.g., similar to the handle region 12). In some instances, the rod may comprise a polypropylene rod with silicone tubing at one end that forms the handle region. The transfer region 14 of the device/rod 10 may be contacted with a suitable broth to allow microorganisms to transfer to the broth. The broth may be monitored for presence of turbidity, signifying the successful transfer of microorganisms to the broth. In some instances, the optical density of the broth may be measured, e.g., at a wavelength of 600 nm, to quantify the concentration of microbial cells. In some instances, the device/rod 10 may be used to transfer the microorganisms to a liquid and, optionally, the liquid containing the microorganisms may then be used to inoculate a target (e.g., a growth medium, e.g., a dehydrated growth medium such as PetriFilm™). In some instances, the device/rod 10 may include a cap (e.g., similar to the cap 28 disclosed herein) that can overlie the transfer region 14. The device/rod 10 may also be packaged in a pouch (e.g., similar to the pouch 30 disclosed herein). In some of these and in other instances, the device/rod 10 may not need a cap (e.g., similar to the cap 28 disclosed herein).

The disclosure is not intended to limit the device 10 to transferring microorganisms to a solid (e.g., agar) target or liquid media as a number of additional uses are also contemplated. In general, the device 10 may be used for growth promotion testing, environmental control/testing, validation testing (e.g., including validating instruments), as a starter culture, to transfer a known microorganism and/or a known quantity of microorganisms, combinations thereof, and/or the like. In some of these and in other instances, the device 10 may be used as a positive control device. For example, the device 10 may be brought into contact with a sampling device (e.g., a microbial collection device) such as a sample collection sponge or swab such as those used for environmental control/testing. In some instances, the sample collection sponge or swab may be pre-wetted (e.g., pre-wetted by the manufacturer) and the pre-wetted sponge or swab may be contacted with the device 10. After contacting the sampling device with the device 10, the microorganisms may be extracted from the sampling device, e.g., using a solution (e.g., a liquid media). In some instances, after contacting the sponge with the device 10, the sponge may be wrung out/squeezed to remove the pre-wetting solution (e.g., a neutralizing buffer) from the sponge and the pre-wetting solution may be captured/recovered. The captured/recovered solution may be tested for the presence of the microorganisms. The presence of microorganisms in the recovered solution serves as a positive control that signifies/verifies that the microorganisms were successfully transferred to the sponge and that the sponge is suitable for collecting microorganisms (e.g., as part of a test such as environmental control/testing). The quantity of microorganisms in the captured/recovered solution may be evaluated or measured (e.g., by enumeration of colony forming units using a culture media).

In some instances, the device 10 may be understood to be a single use or “disposable” device. In other instances, the device 10 may be understood to a multi-use or reusable device.

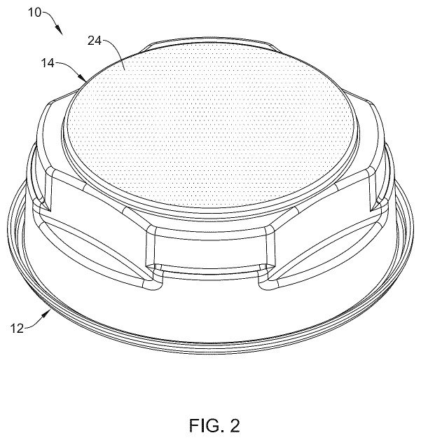

As indicated above, the pre-determined quantity of viable microorganisms, schematically depicted in FIG. 2 and denoted with reference number 24, may be disposed on the transfer region 14. In some instances, the pre-determined quantity of viable microorganisms 24 are coated onto the transfer region 14 and then air dried (e.g., the microorganisms are air-dried). In some of these and in other instances, the microorganisms 24 may be freeze-dried, lyophilized, dehydrated, combinations thereof, and/or the like. In some instances, about 1 to 1000 colony forming units of viable microorganisms 24 may be disposed along the transfer region 14, or about 1 to 100 colony forming units of viable microorganisms 24 may be disposed along the transfer region 14.

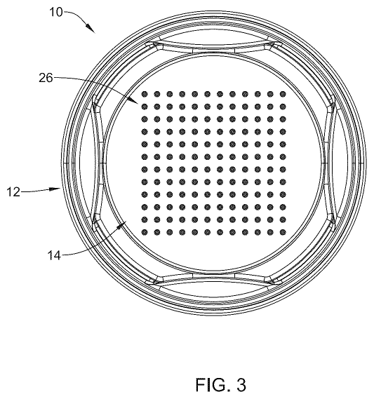

In some instances, the microorganisms 24 may be disposed evenly/homogeneously along the transfer region 14. In some of these and in other instances, the microorganisms 24 may disposed/arranged in a pattern along the transfer region 14. For example, the pattern may include an array of microbial clusters 26 as depicted in FIG. 3. In some instances, the microbial clusters 26 may each include one or more microbial cells that are designed to produce a distinct colony forming unit (e.g., when the transfer region 14 is brought into contact with the target). This may allow for the efficient transfer of discrete colonies/clusters that can be easily visualized by a user following the transfer (e.g., after a suitable incubation period the colonies/clusters can be visualized in the pattern).

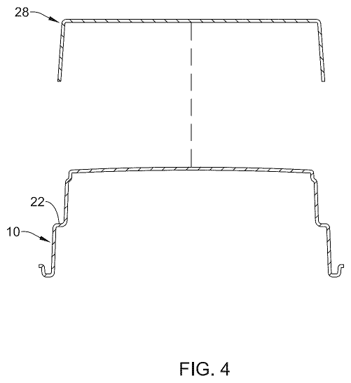

In some instances, a cap 28 may be disposed over the transfer region 14 as schematically depicted in FIGS. 4-5. For example, after disposing the microorganisms 24 onto the transfer region 14, the cap 28 may be placed over the transfer region 14. In some instances, this may include placing the edge or lip of the cap 28 along the cap receiving region 16. More particularly, the edge or lip of the cap 28 may be disposed within the grooved region 22. In some instances, the spacer sections 20 may allow for some air flow to occur along the cap receiving region 16. In addition, when the device 10 and cap 28 are disposed within a package/pouch/container (e.g., as described herein), the spacer sections 20 may allow for the atmosphere and/or conditions within the pouch to have access to the microorganisms 24. In some instances, the cap 28 may include a mechanism or system that secures the cap to the device 10. For example, the cap 28 may be threaded and/or have a locking mechanism in conjunction with the device 10.

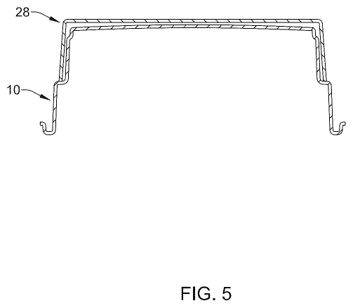

Once the cap 28 is disposed over the transfer region 14, the device 10 (having the microorganisms 24 along the transfer region 14) may be disposed in a package or pouch 30 as shown in FIG. 6. The package 30 may include airflow and/or moisture resistant materials that are generally designed to control the flow of air and/or moisture into and out from the package 30. For example, the package 30 may include a metal film, metal foil, polymer film (e.g., a stretch or biaxially-oriented polyethylene terephthalate film such as MYLAR), combinations thereof, and/or the like. In some instances, a particular atmosphere such as a modified atmosphere where a gas (e.g., nitrogen) may be used within the package 30. Other atmospheres and/or conditions may be utilized.

In some instances, a desiccant and/or oxygen scavenger may be disposed within the package 30. The quantities of desiccant and/or oxygen scavenger used may help to provide a suitable environment for the microorganisms 24 to have the desired viability. For example, a relatively small amount of desiccant may be added to the package 30. As such, the relative humidity within the package 30 may be 10% or more, or about 20% or more, or about 30% or more, or about 20-30% or more. In addition, a relatively large amount of oxygen scavenger may be added to the package 30. As such, the oxygen level/content within the package may be relatively low. For example, the oxygen content within the package may be about 10% or less, or about 5% or less, or about 4% or less, or about 3% or less, or about 2% or less, or about 1% or less.

The blend of relatively high relative humidity and relatively low oxygen within the package 30 surprising resulted in enhanced viability of the microorganisms 24. For example, a package 30 as described herein has been shown to result in 99% or more viability when stored at an elevated temperature (97° F.) for greater than 90 days. In some instances, the microorganisms 24 are room temperature stable. In some instances, the microorganisms 24 are stable at a temperature of 20° C. or less. In some instances, the microorganisms 24 are stable at a temperature of 4° C. or less. In some instances, the microorganisms 24 have a viability of 50% or more after 30 days. In some instances, the microorganisms 24 have a viability of 50% or more after 90 days. In some instances, the microorganisms 24 have a viability of 50% or more at 20° C. for 90 days. In some instances, the microorganisms 24 have a viability of 75% or more at 20° C. for 90 days. In some instances, the microorganisms 24 have a viability of 90% or more at 20° C. for 90 days.

To use the device 10, the device may be removed from the package 30. This may include cutting open or otherwise unsealing the package. Upon removal, the cap 28 may be removed and the transfer region 14 may be brought into contact with the target. As discussed herein, an additional step of rehydrating the microorganisms 24 prior to the inoculating/transferring step is not required.

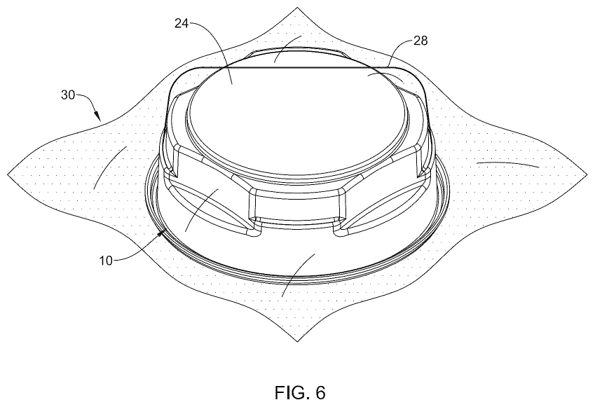

FIG. 7 illustrates another illustrative inoculating device 110 that may be similar in form and function to other inoculating devices disclosed herein. The inoculating device 110 may include a handle region 112 and a transfer region 114. A plurality of projections 132 may be disposed along the transfer region 114. The microorganisms (e.g. the microorganisms 24) may be disposed along the projections 132 (e.g., along one or more of the projections 132). In some instances, the microorganisms may only be disposed along the projections 132 (e.g., along the top or end surfaces of the projections 132). In some of these instances, the remainder of the transfer region 114 may be free of the microorganisms. In other instances, the microorganisms may be disposed along both the projections 132 and along other parts of the transfer region 114 (e.g., parts that do not include projections 132).

In some instances, one or more of the projections 132 may have a convex shape and/or otherwise include a surface that is convex. For example, the top or end of the projections 132 may bow outward or otherwise take the form of a convex surface. In some of these and in other instances, one or more of the projections 132 may have a planar shape and/or otherwise includes a surface that is planar. For example, the top or end of the projections 132 may be flat or otherwise take the form of a planar surface. In addition, the remainder of the transfer region 114 may be convex or planar. Thus, in some instances the transfer region 114 may bow outward or otherwise take the form of a convex surface, projections 132 may be disposed along the transfer region 114, and the ends of the projections 132 may also bow outward or otherwise take the form of a convex surface.

The transfer region 114 may include a suitable number of projections 132. For example, the transfer region 114 may include about 2 to 1,000 projections 132, or about 2 to 100 projections 132, or about 2 to 50 projections 132, or about 40 projections. The projections 132 may also have a suitable size. For example, the diameter or a diagonal across the projections 132 may be about 1 mm or more, or about 1 to 25 mm or more, or about 1 to 10 mm or more, or about 1 to 5 mm. The surface area of the projections 132 (e.g., the surface area of the end surface) may be about 0.5 mm² or more, or about 0.5 to 1,000 mm², or about 0.5 to 20 mm². In some instances, the sides or peripheral surfaces of the projections 132 may be rounded, blunt, unsharp, or the like. The projections 132 may have a suitable height. For example, the projections 132 may have a height of 5 mm or less, 4 mm or less, 3 mm or less, 2 mm or less, or 1 mm or less.

The use of a transfer region 114 with projections 132 may be desirable for a number of reasons. For example, the projections 132 may help to reduce the amount of contact between the transfer region 114 and the target. This may help to reduce suction or binding between the transfer region 114 and the target during transfer. For example, if the transfer region 114 was to bind with the target, pulling the transfer region 114 away from the target could bend or deflect the target material. This may result in an uneven or imprecise transfer of microorganisms and/or result in challenges with spreading microorganisms evenly across the target. Thus, the projections 132 may help to reduce/avoid these potential drawbacks during transfer.

Forming the inoculating device 110 (e.g., including the transfer region 114 with projections 132) may include those processes and materials disclosed herein. For example, the inoculating device 110 may be formed using thermoforming, injection molding, molding, casting, and/or the like. In some instances, the inoculating device 110 may be made from materials such as polypropylene, polyethylene, polyethylene terephthalate, glycol modified polyethylene terephthalate, polystyrene, poly-lactic acid, acrylonitrile butadiene styrene, polyetheretherketone, polyoxymethylene, nylon, polycarbonate, polytetrafluoroethylene, or combinations thereof. In one example, the inoculating device 110 may be formed from polypropylene (e.g., via an injection molding process).

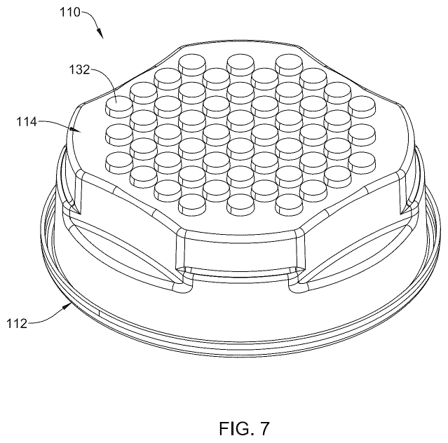

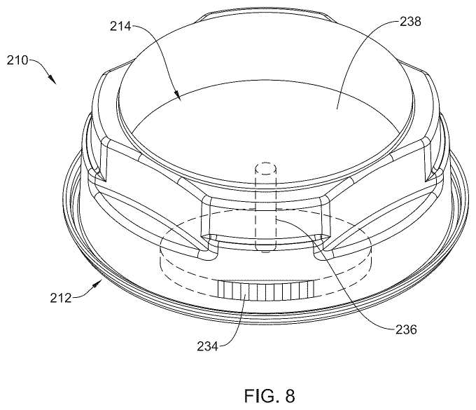

FIGS. 8-9 illustrate another illustrative inoculating device 210 that may be similar in form and function to other inoculating devices disclosed herein. The inoculating device 210 may include a handle region 212 and a transfer region 214. In this example, the transfer region 214 may include a moveable platform 238 coupled to an actuator 236 by a rod 236. Actuation of the actuator 236 (e.g., rotation of the actuator 236) may result in the platform 238 raising and lowering. For example, when in a storage configuration such as that shown in FIG. 8, the platform may be recessed or otherwise disposed closer to the handle region 212. When the actuator 236 is actuated, the platform 238 may shift toward a transfer configuration where the platform 238 and/or the transfer region 214 can be utilized to transfer microorganisms to a target. The process for disposing the microorganisms 24 onto the transfer region 14 may include a number of steps. For example, a cell suspension may be prepared with a known quantity and/or concentration of microorganisms.

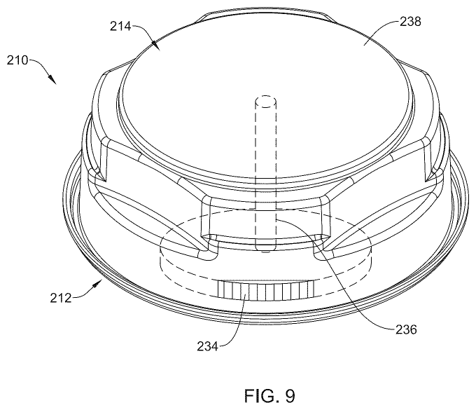

FIG. 10 is a perspective view of an illustrative inoculating device 300 that may be used to transfer viable microorganisms to a target that may include a solid target or a liquid target. In some cases, the target may be a microbial collection device. The inoculating device 300 includes an elongate body 302. The elongate body 302 may be formed in any suitable matter, and out of any suitable material. In some cases, the elongate body 302 may be an elongated polymeric body. The elongate body 302 may be formed via injection molding, as an example. The elongate body 302 may be formed of polypropylene or polycarbonate, for example, although other polymeric materials are contemplated.

The inoculating device 302 includes a transfer region 304, which as shown may be disposed on a distal end 306 of the elongate body 302. The transfer region 304 may be adapted to accommodate a solution 307 that has been coated and dried onto the transfer region 304 that includes viable microorganisms. The viable microorganisms used may be any of those described below.

The inoculating device 302 includes a cover receiving region 308 that may be considered as encompassing the transfer region 304. In some case, the cover receiving region 308 may include a securement region 310 that serves to releasably secure a cover (not shown in FIG. 10) The cover receiving region 308 and the securement region 310 may together be adapted to accommodate a cover that is formed as a cylindrical or substantially cylindrical shape, with one open end that engages with the securement region 310 and a closed end that covers (but does not contact) the transfer region 306. The cover, when secured in position, protects the transfer region 306 (as well as the coating 307 that includes the viable microorganisms). The cover, when secured in position, may hold a modified atmosphere such as but not limited to an inert gas. The cover, when secured in position, may hold a desiccant or an oxygen scavenger, for example.

The inoculating device 302 includes a handle region 312 that allows a user to grasp and manipulate the inoculating device 302. In some cases, the handle region 312 may include a grip section 314 that is adapted to be easy for a user to grasp the grip section 314 using their fingertips, especially when the user is wearing gloves, for example. The grip section 314 may be formed as part of the handle region 312 when the elongate body 302 is formed, such as via injection molding. The handle region 312 may include a number of flutes 316. In some cases, the flutes 316 allow less total material to be used in forming the handle region 312. In some cases, the flutes 316 allow for other advantages such as allowing mechanical grasping of the inoculation device 300 during manufacturing.

The elongate body 302 may have an overall length of about 10 cm to about 30 cm. The elongate body 302 may have an overall length of about 20 cm. The elongate body 302 may have a diameter of about 0.5 cm to about 1.5 cm. The elongate body 302 may have a diameter that is about 1 cm. It will be appreciated that the diameter of the elongate body 302 may vary, depending on exactly where along the length of the elongate body 302 the diameter is measured. In some cases, the cover region 308 may have an overall length that is in a range of about 10 percent to about 60 percent of the overall length of the elongate body 302. In some cases, the handle region 312 may have an overall length in a range of about 40 percent to about 90 percent of the overall length of the elongate polymeric body. These are just examples.

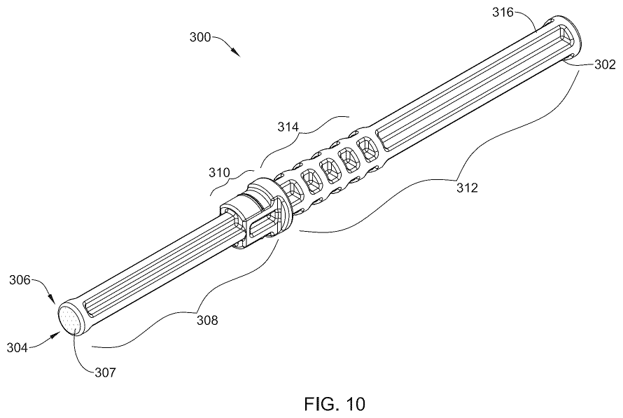

FIG. 11 shows the inoculating device 300 with a cover 318 shown disposed over the transfer region 306 and the cover receiving region 308, and forming an interference fit with the securement region 310. The cover 318 may be considered as being largely cylindrical in shape, with an open end 320 where the cover 318 interacts with the securement region 310, and a closed end 322 located opposite the open end 320. The closed end 322 may be considered as enveloping the transfer region 306 but not contacting the transfer region 306 or the coating 307 formed on the transfer region 306. The cover 318 may be formed of any suitable material, such as a polymeric material. In some cases, the cover 318 may be formed of a transparent polymer that allows viewing of the transfer region 306. In some cases, the cover 318 may be adapted to allow for a sticker or other insignia to be placed on the cover 318 that identifies the viable microorganisms within the coating 307 formed on the transfer region 306, or other pertinent information.

The inoculating device 300 is shown disposed within a moisture barrier container 320. The moisture barrier container 320 may be adapted to resist the flow of water vapor from a position outside the moisture barrier container 320 to a position inside the moisture barrier container 320, or from a position inside the moisture barrier container 320 to a position outside the moisture barrier container 320. In some cases, the moisture barrier container 320 includes a container 322 and a removable cover 324 that is releasably securable to the container 322. In some cases, the removable cover 324 may be adapted to be peeled off of the container 322 in order to access an interior of the moisture barrier container 320 and thus access the inoculation device 300. In some cases, the removable cover 324 may be a foil cover, for example. The removable cover 324 may also include text, images or other insignia identifying the contents of the moisture barrier container 320 and/or providing storage or use instructions.

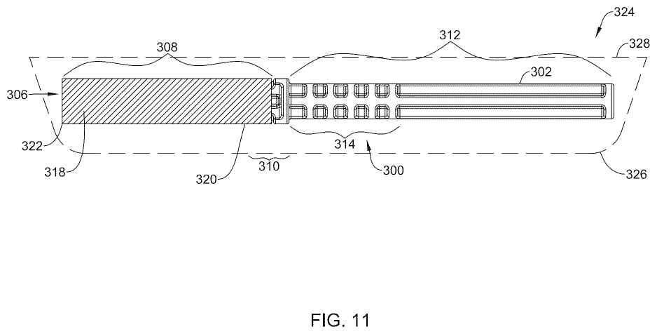

FIG. 12 is a side view of an illustrative inoculation device 330 that may be used in transferring viable microorganisms from a dried coating that includes the viable microorganisms to a liquid target that can be placed within the inoculation device 330. The inoculation device 330 includes a vessel 332. The vessel 332 has an open end 334 and a closed end 336. A cap 338 is adapted to be releasably secured to the open end 334. In some cases, the cap 338 may be adapted to form a mechanical fit such as an interference fit with the open end 334. In some cases, the cap 338 may be adapted to thread onto the open end 334. Alternatively, the cap 338 may instead be adapted to fit inside the open end 334, such as a rubber stopper having a tapered or even frustroconical shape.

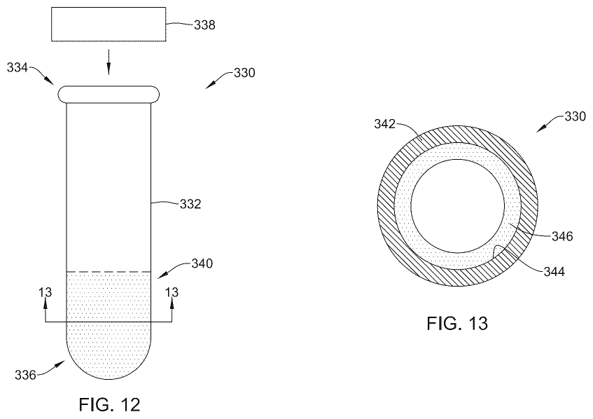

In some instances, the vessel 332 may be considered as being similar to a test tube. The vessel 332 may be formed of any suitable material, such as a polymer or even glass. In some cases, the vessel 332 may be transparent. The inoculation device 330 has a transfer region 340 disposed within an inside of the vessel 332. FIG. 13 is a cross-sectional view of the inoculation device 330, taken along the line 13-13 of FIG. 12. As can be seen in the cross-sectional view, the inoculation device 330 includes a vessel wall 342 that defines the vessel 332. A dried coating 346 is shown on an inside surface 344 of the vessel wall 342. The dried coating 346 may include a variety of different viable microorganisms, such as those listed below.

In some instances, of any of the methods or articles provided herein, the target may be a solid media or a liquid solution. In other instances, the target may be a microbial collection device. Provided herein are systems, devices, and methods for rapidly transferring viable microorganisms to a target (e.g., any of the targets described herein) from an inoculating member in a dry state without the need for an additional step to prepare the inoculum. In some cases, the inoculating systems provided herein are useful for storing viable microorganisms and rapidly transferring the viable microorganisms to a liquid solution. In some cases, the inoculating systems provided herein are useful for storing viable microorganisms and rapidly inoculating a solid media (e.g., an agar plate or a dry media sheet). In some instances, the inoculating systems provided herein are useful for storing viable microorganisms and rapidly transferring the viable microorganisms to a microbial collection device (e.g., a swab, sponge, or contact plate). In some cases, a quantity (e.g., a pre-defined quantity) of viable microorganisms are transferred to a target. In some instances, the inoculating system or device may be disposable. It will be appreciated that in any of the inoculating systems or devices provided herein, the viable microorganisms define an inoculum.

As noted, the target may be a solid media or a liquid solution. In other cases, the target may be a microbial collection device (e.g., a swab, a sponge, or a contact plate). Examples of solid media and liquid solution targets capable of accepting biological material (e.g., any of the inoculums provided herein) are known in the art. Examples of microbial collection devices are known in the art.

In some cases, the inoculating member may be configured to contact a solid target. In some instances, the inoculating member may be configured to directly contact a solid target (e.g., a solid media such as agar media or dry sheet media) with the transfer region. In some cases, the transfer region may extend from the handle region of the inoculating system. In some cases, the transfer region may be configured to reduce or minimize or eliminate pooling, clustering, or grouping of viable microorganisms upon transfer to a solid target. Other examples of solid targets capable of accepting biological material (e.g., any of the inoculums provided herein) are known in the art.

In some cases, the inoculation device may be configured to receive a liquid (e.g. a liquid target. In some cases, the inoculation device may be configured to receive a liquid (e.g., a liquid target) and submerge the transfer region with the liquid. In some cases, the inoculation device may include a tube shape with one closed end and one open end. In some cases, the transfer region may be located within a tube or vial. In some cases, the viable microorganisms are dried within a tube or vial. Examples of liquid targets include distilled water, phosphate buffer solution, and liquid growth media. Other examples of liquid targets capable of accepting biological material (e.g., any of the inoculums provided herein) are known in the art.

In some cases, the inoculating member may be adapted to store a quantity of liquid (e.g., about 50 mL or less, about 45 mL or less, about 40 mL or less, about 35 mL or less, about 30 mL or less, about 25 mL or less, about 20 mL or less, about 15 mL or less, about 10 mL or less, about 5 mL or less, about 3 mL or less, about 2 mL or less, about 1 mL or less, about 950 μL or less, about 900 μL or less, about 850 μL or less, about 800 μL or less, about 750 μL or less, about 700 μL or less, about 650 μL or less, about 600 μL or less, about 550 μL or less, about 500 μL or less, about 450 μL or less, about 400 μL or less, about 350 μL or less, about 300 μL or less, about 250 μL or less, about 200 μL or less, about 150 μL or less, about 100 μL or less, or about 50 μL or less) in order to facilitate transfer of the inoculum to a liquid target.

In some cases, the target may be a microbial collection device (e.g., a swab, a sponge, or a contact plate). Examples of microbial collection devices capable of accepting biological material (e.g., any of the inoculums provided herein) are known in the art. The inoculation device (e.g., any of the inoculating systems provided herein) can be configured to receive a microbial collection tool. In some cases, the inoculating member may be configured to be used as a substrate for performing a method of microbial collection (e.g., using a microbial collection device). In some examples, the inoculation device may be configured to transfer the viable microorganisms to a microbial collection device.

In some cases, the inoculation device may include a flat or planar surface. In some cases, the transfer region includes a flat or planar surface. The transfer region may be a rough surface. The transfer region may have a defined surface roughness. In some cases, the transfer region may have a surface area of between about 25 cm² and about 900 cm². The transfer region may have a surface area of between about 50 cm² and about 225 cm², for example. In some cases, the inoculation device does not require an elevated temperature to transfer the viable microorganisms, and in some cases, may be adapted to transfer the viable microorganisms to a solid growth media, a liquid solution, or a microbial collection device at a temperature between about 15° C. to about 25° C.

In some cases, the inoculation device may be configured to transfer the viable microorganisms to a solid growth media, a liquid solution, or a microbial collection device in 60 seconds or less, 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 3 seconds or less, 2 seconds or less, or 1 second or less at a temperature between about 15° C. to about 25° C. In some cases, the inoculation device may be configured to transfer the viable microorganisms to a solid growth media, a liquid solution, or a microbial collection device in 30 seconds or less at a temperature between about 15° C. to about 25° C. In some cases, the inoculation device may be configured to transfer the viable microorganisms to a solid growth media, a liquid solution, or a microbial collection device in 10 seconds or less at a temperature between about 15° C. to about 25° C. In some cases, the inoculation device may be configured to transfer the viable microorganisms to a solid growth media, a liquid solution, or a microbial collection device in 5 seconds or less at a temperature between about 15° C. to about 25° C. The viable microorganisms may be substantially transferred to the target in 60 seconds or less 60 seconds or less, 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 3 seconds or less, 2 seconds or less, or 1 second or less (e.g., at a temperature between about 15° C. to about 25° C.).

In some cases, the inoculation device includes a transfer region that is adapted to be inserted at least 1 mm, at least 2 mm, at least 3 mm, at least 4 mm, at least 5 mm, at least 6 mm, at least 7 mm, at least 8 mm, at least 9 mm, at least 1 cm, at least 2 cm, at least 3 cm, at least 4 cm, at least 5 cm, at least 6 cm, at least 7 cm, at least 8 cm, at least 9 cm, at least 10 cm, or greater than 10 cm within a tube or vial. In some cases, the inoculation device may include an extension region proximal of the transfer region that is adapted to be inserted within a tube or vial without introducing contamination.

In some cases, the inoculation device may be configured to transfer at least 50%, at least 60%, at least 70%, at least 75%, at least 80%, at least 85%, at least 90%, at least 95%, at least 99% or 100% of the viable microorganisms to a solid growth media, a liquid solution, or a microbial collection device in 60 seconds or less, 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 3 seconds or less, 2 seconds or less, or 1 second or less at a temperature between about 15° C. to about 25° C.

The inoculation device may be configured to transfer at least 99% or more of the viable microorganisms to a solid growth media, a liquid solution, or a microbial collection device in 30 seconds or less at a temperature between about 15° C. to about 25° C. The inoculation device may be configured to transfer at least 99% or more of the viable microorganisms to a solid growth media, a liquid solution, or a microbial collection device in 10 seconds or less at a temperature between about 15° C. to about 25° C. The inoculation device may be configured to transfer at least 99% or more of the viable microorganisms to a solid growth media, a liquid solution, or a microbial collection device in 5 seconds or less at a temperature between about 15° C. to about 25° C. The inoculation device may be configured to transfer 100% of the viable microorganisms to a solid growth media, a liquid solution, or a microbial collection device in 30 seconds or less at a temperature between about 15° C. to about 25° C. The inoculation device may be configured to transfer 100% of the viable microorganisms to a solid growth media, a liquid solution, or a microbial collection device in 10 seconds or less at a temperature between about 15° C. to about 25° C. The inoculation device may be configured to transfer 100% of the viable microorganisms to a solid growth media, a liquid solution, or a microbial collection device in 5 seconds or less at a temperature between about 15° C. to about 25° C.

In some instances, the inoculation device may be configured to transfer the viable microorganisms to a solid media or a liquid solution at a transfer rate, wherein the transfer rate is at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 99% of the total viable microorganisms per second at a temperature between about 15° C. to about 25° C. The inoculation device may be configured to transfer the viable microorganisms to a solid growth media, a liquid solution, or a microbial collection device at a transfer rate of at least about 1 CFU, about 10 CFU, about 25 CFU, about 50 CFU, about 100 CFU, about 150 CFU, about 200 CFU, about 250 CFU, about 300 CFU, about 350 CFU, about 400 CFU, about 450 CFU, about 500 CFU, about 550 CFU, about 600 CFU, about 650 CFU, about 700 CFU, about 750 CFU, about 800 CFU, about 850 CFU, about 900 CFU, about 950 CFU, about 1,000 CFU, about 1,500 CFU, about 2,000 CFU, about 2,500 CFU, about 5,000 CFU, about 10,000 CFU, about 100,000 CFU, about 1,000,000 CFU, about 10,000,000 CFU, about 100,000,000 CFU, about 1,000,000,000 CFU per second at a temperature between about 15° C. to about 25° C.

In some cases, the quantity of microorganisms transferred to a solid growth media, a liquid solution, or a microbial collection device may be within a desired range of a lower limit and an upper limit of viable microorganisms. In some cases, the desired range of viable microorganisms transferred to a target (e.g., any of the targets provided herein) may include a lower limit and an upper limit between about 1 and 10⁹ colony forming units, about 1 and 100 colony forming units, about 100 and 1,000 colony forming units, or about 100 and 2,000 colony forming units. The lower limit and upper limit of the desired range of viable microorganisms transferred to a target are each selected from a value between about 1 to about 10⁹ colony forming units, for example. The lower limit and upper limit of the desired range of viable microorganisms transferred to a target may each be selected from a value between about 10⁵ to about 10⁹ colony forming units. The lower limit and upper limit of the desired range of viable microorganisms transferred to a target may each be selected from a value between about 10⁷ to about 10⁹ colony forming units.

In some cases, the inoculation device may be stored within a moisture barrier container, and the moisture barrier container may be further sealed within a secondary container. The moisture barrier container may include one or more of a modified atmosphere, a desiccant, an oxygen scavenger, or an inert gas.

A cap receiving region may include a mechanism to enable airflow between the transfer region and the packaging environment of the moisture barrier container. In some cases, a cap may include a vent or opening to enable airflow between the transfer region and the packaging environment of the moisture barrier container. Contained within the cap may be one or more of a modified atmosphere, a desiccant, or an oxygen scavenger. In some cases, the inoculation device or portions thereof may be sterile. The extension region, if present, may be sterile. The transfer region may be free of contaminants. The extension region may be free from contaminants.

In some cases, the inoculation device may include an elongated member such as an elongated polymeric body. The inoculation device may have a rod shape, for example. In some cases, the inoculation device may have a length of about 30 cm or less, about 25 cm or less, about 20 cm or less, about 15 cm or less, about 14 cm or less, about 13 cm or less, about 12 cm or less, about 11 cm or less, about 10 cm or less, about 9 cm or less, about 8 cm or less, about 7 cm or less, about 6 cm or less, about 5 cm or less, about 4 cm or less, about 3 cm or less, about 2 cm or less, or about 1 cm or less. In some cases, the inoculation device may a length of greater than about 30 cm.

In some cases, the inoculation device may have a width of about 30 cm or less, about 25 cm or less, about 20 cm or less, about 15 cm or less, about 14 cm or less, about 13 cm or less, about 12 cm or less, about 11 cm or less, about 10 cm or less, about 9 cm or less, about 8 cm or less, about 7 cm or less, about 6 cm or less, about 5 cm or less, about 4 cm or less, about 3 cm or less, about 2 cm or less, about 1 cm or less, 9 mm or less, about 8 mm or less, about 7 mm or less, about 6 mm or less, about 5 mm or less, about 4 mm or less, about 3 mm or less, about 2 mm or less, about 1 mm or less. In some cases, the inoculation device may have a width of greater than about 30 cm.

In some cases, the cap receiving region of the inoculation device may have a length of about 15 cm or less, about 14 cm or less, about 13 cm or less, about 12 cm or less, about 11 cm or less, about 10 cm or less, about 9 cm or less, about 8 cm or less, about 7 cm or less, about 6 cm or less, about 5 cm or less, about 4 cm or less, about 3 cm or less, about 2 cm or less, about 1 cm or less, 9 mm or less, about 8 mm or less, about 7 mm or less, about 6 mm or less, about 5 mm or less, about 4 mm or less, about 3 mm or less, about 2 mm or less, about 1 mm or less. In some cases, the cap receiving region or transfer region may have a length of greater than about 30 cm.

In some cases, the cap receiving region or transfer region may have a width of about 15 cm or less, about 14 cm or less, about 13 cm or less, about 12 cm or less, about 11 cm or less, about 10 cm or less, about 9 cm or less, about 8 cm or less, about 7 cm or less, about 6 cm or less, about 5 cm or less, about 4 cm or less, about 3 cm or less, about 2 cm or less, about 1 cm or less, 9 mm or less, about 8 mm or less, about 7 mm or less, about 6 mm or less, about 5 mm or less, about 4 mm or less, about 3 mm or less, about 2 mm or less, about 1 mm or less. In some cases, the cap receiving region or transfer region may have a width of greater than about 30 cm.

In some cases, the handle region may have a length of about 20 cm or less, about 15 cm or less, about 14 cm or less, about 13 cm or less, about 12 cm or less, about 11 cm or less, about 10 cm or less, about 9 cm or less, about 8 cm or less, about 7 cm or less, about 6 cm or less, about 5 cm or less, about 4 cm or less, about 3 cm or less, about 2 cm or less, or about 1 cm or less. In some cases, the handle region may havbe a width of about 5 cm or less, about 4 cm or less, about 3 cm or less, about 2 cm or less, or about 1 cm or less.

In some cases, the transfer region may have a length of about 30 cm or less, about 25 cm or less, about 20 cm or less, about 15 cm or less, about 14 cm or less, about 13 cm or less, about 12 cm or less, about 11 cm or less, about 10 cm or less, about 9 cm or less, about 8 cm or less, about 7 cm or less, about 6 cm or less, about 5 cm or less, about 4 cm or less, about 3 cm or less, about 2 cm or less, about 1 cm or less, 9 mm or less, about 8 mm or less, about 7 mm or less, about 6 mm or less, about 5 mm or less, about 4 mm or less, about 3 mm or less, about 2 mm or less, about 1 mm or less. In some cases, the transfer region may have a length of greater than about 30 cm.

In some cases, the transfer region may have a width of about 30 cm or less, about 25 cm or less, about 20 cm or less, about 15 cm or less, about 14 cm or less, about 13 cm or less, about 12 cm or less, about 11 cm or less, about 10 cm or less, about 9 cm or less, about 8 cm or less, about 7 cm or less, about 6 cm or less, about 5 cm or less, about 4 cm or less, about 3 cm or less, about 2 cm or less, about 1 cm or less, 9 mm or less, about 8 mm or less, about 7 mm or less, about 6 mm or less, about 5 mm or less, about 4 mm or less, about 3 mm or less, about 2 mm or less, about 1 mm or less. In some cases, the transfer region may have a width of greater than about 30 cm.

In some cases, the transfer region may have a surface area of about 4,000 cm² or less (e.g., about 3,500 cm² or less, about 3,000 cm² or less, about 2,500 cm² or less, about 2,000 cm² or less, about 3,500 cm² or less, about 3,000 cm² or less, about 2,500 cm² or less, about 2,000 cm² or less, about 1,500 cm² or less, about 1,000 cm² or less, about 950 cm² or less, about 900 cm² or less, about 850 cm² or less, about 800 cm² or less, about 750 cm² or less, about 700 cm² or less, about 650 cm² or less, about 600 cm² or less, about 550 cm² or less, about 500 cm² or less, about 450 cm² or less, about 400 cm² or less, about 350 cm² or less, about 300 cm² or less, about 250 cm² or less, about 200 cm² or less, about 150 cm² or less, about 100 cm² or less, about 90 cm² or less, about 80 cm² or less, about 70 cm² or less, about 60 cm² or less, about 50 cm² or less, about 40 cm² or less, about 30 cm² or less, about 20 cm² or less, about 10 cm² or less, about 950 mm² or less, about 900 mm² or less, about 850 mm² or less, about 800 mm² or less, about 750 mm² or less, about 700 mm² or less, about 650 mm² or less, about 600 mm² or less, about 550 mm² or less, about 500 mm² or less, about 450 mm² or less, about 400 mm² or less, about 350 mm² or less, about 300 mm² or less, about 250 mm² or less, about 200 mm² or less, about 150 mm² or less, about 100 mm² or less, about 90 mm² or less, about 80 mm² or less, about 70 mm² or less, about 60 mm² or less, about 50 mm² or less, about 40 mm² or less, about 30 mm² or less, about 20 mm² or less, about 10 mm² or less, about 9 mm² or less, about 8 mm² or less, about 7 mm² or less, about 6 mm² or less, about 5 mm² or less, or about 4 mm² or less). In other cases, the transfer region may have a surface area of greater than about 4,000 cm².

In some case, a maximum thickness of the material forming any of the handle region, the cap receiving region, the extension region, or the transfer region may be about 10 mm or less, about 9 mm or less, about 8 mm or less, about 7 mm or less, about 6 mm or less, about 5 mm or less, about 4 mm or less, about 3 mm or less, about 2 mm or less, or about 1 mm or less. In some cases, a maximum thickness of any material forming the inoculating member is about 10 mm or less, about 9 mm or less, about 8 mm or less, about 7 mm or less, about 6 mm or less, about 5 mm or less, about 4 mm or less, about 3 mm or less, about 2 mm or less, or about 1 mm or less.

In some cases, the inoculating system may not include a handle region. The transfer region may include a solid substrate. The transfer region may be a flat surface or a substantially planar surface. The transfer region may have a convex surface or a paddle shape. The transfer region may be orientated perpendicular to the handle region. The transfer region may be orientated parallel to the handle region. The transfer region may be orientated at an angle between 0° and 90° compared to the handle region.

In some cases, the inoculation device may be disposable. The inoculation device may be formed of or otherwise include a plastic. The inoculation device may be formed of a plastic such as polypropylene or polycarbonate. In some cases, the transfer region may be compatible with drop coating and drying of a coating solution that includes viable microorganisms onto the transfer region. In some cases, the transfer region may be adapted to enable drop coating and drying of a coating solution including viable microorganisms onto the transfer region. The viable microorganisms dried on the transfer region include a desired quantity of viable microorganisms, wherein the desired quantity of viable microorganisms includes a desired lower limit and a desired upper limit of colony forming units.

In some cases, the viable microorganisms dried on the transfer region stabilize within a desired lower limit and a desired upper limit of colony forming units after a period between about 1 hour and about 30 days and at a temperature of 2-8° C., or a temperature of 15-25° C. The viable microorganisms dried on the transfer region may stabilize within a desired lower limit and desired upper limit of colony forming units after a period of about 1 hour, about 2 hours, about 3 hours, about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 22 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days.

In some cases, the viable microorganisms dried on the transfer region may stabilize within a desired lower limit and a desired upper limit of colony forming units after a period between about 30 days and about 90 days and at a temperature of 2-8° C. or a temperature of 15-25° C. The viable microorganisms dried on the transfer region may stabilize within a desired lower limit and a desired upper limit of colony forming units after a period of greater than about 90 days and at a temperature of 2-8° C. or a temperature of 15-25° C. After the viable microorganisms stabilize within a desired lower limit and a desired upper limit of colony forming units, the viable microorganisms may remain stabilized within the desired lower limit and the desired upper limit for a period between about 30 days and about 2 years and at a temperature of 2-8° C. or a temperature of 15-25° C.

After the viable microorganisms stabilize within the desired lower limit and desired upper limit of colony forming units, the viable microorganisms may remain stabilized within the desired lower limit and desired upper limit of colony forming units for a period of at least about 30 days, about 60 days, about 90 days, about 120 days, about 150 days, about 180 days, about 210 days, about 240 days, about 270 days, about 300 days, about 350 days, about 365 days, about 400 days, about 450 days, about 500 days, about 550 days, about 600 days, about 650 days, about 700 days, or about 730 days after sealing the inoculation device in the moisture barrier container.

In some cases, after the viable microorganisms stabilize within a desired lower limit and a desired upper limit of colony forming units, the viable microorganisms remain stabilized within the desired lower limit and the desired upper limit for a period greater than about 2 years and at a temperature of 2-8° C. or a temperature of 15-25° C. In some cases, after the viable microorganisms stabilize within a desired lower limit and a desired upper limit of colony forming units, the viable microorganisms may remain stabilized within the desired lower limit and the desired upper limit for a period of at least 1 year, at least 2 years, or at least 3 years and at a temperature of 2-8° C. or a temperature of 15-25° C.

In some cases, the desired range of viable microorganisms includes a lower limit and an upper limit of colony forming units that remain viable 30 days after sealing the inoculation device in the moisture barrier container at a temperature of 2-8° C. or a temperature of 15-25° C. In some cases, the desired range of viable microorganisms remain stably within the lower limit and the upper limit of colony forming units for at least 90 days after sealing the inoculation device in the moisture barrier container at a temperature of 2-8° C. or a temperature of 15-25° C. In some cases, the desired range of viable microorganisms remain stably within the lower limit and the upper limit of colony forming units for at least 180 days after sealing the inoculation device in the moisture barrier container at a temperature of 2-8° C. or a temperature of 15-25° C.

In some cases, the desired range of viable microorganisms remain stably within the lower limit and the upper limit of colony forming units for a period of at least 30 days, at least 60 days, at least 90 days, at least 120 days, at least 150 days, at least 180 days, at least 210 days, at least 240 days, at least 270 days, at least 300 days, at least 350 days, at least 365 days, at least 400 days, at least 450 days, at least 500 days, at least 550 days, at least 600 days, at least 650 days, at least 700 days, or at least 730 days after sealing the inoculation device in the moisture barrier container at a temperature of 2-8° C. or a temperature of 15-25° C.

The viable microorganisms stabilize within a desired lower limit and a desired upper limit of colony forming units, and the values for the desired lower limit and the desired upper limit are between about 1 colony forming unit and about 10¹⁰ colony forming units. In some cases, the viable microorganisms stabilize within a desired lower limit and a desired upper limit of colony forming units of between about 1 colony forming unit and about 10¹⁰ colony forming units (e.g., about 1 colony forming unit and about 10⁹ colony forming units, about 1 colony forming unit and about 10⁸ colony forming units, about 1 colony forming unit and about 10⁷ colony forming units, about 1 colony forming unit and about 10⁶ colony forming units, about 1 colony forming unit and about 10⁵ colony forming units, about 1 colony forming unit and about 10⁴ colony forming units, about 1 colony forming unit and about 10³ colony forming units, about 1 colony forming unit and about 10² colony forming units, about 1 colony forming unit and about 10¹ colony forming units, about 1 colony forming unit and about 5,000 colony forming units, about 1 colony forming unit and about 4,000 colony forming units, about 1 colony forming unit and about 3,000 colony forming units, about 1 colony forming unit and about 2,500 colony forming units, about 1 colony forming unit and about 2,000 colony forming units, about 1 colony forming unit and about 1,500 colony forming units, about 1 colony forming unit and 1,250 colony forming units, about 1 colony forming unit and about 1,000 colony forming units, about 10 colony forming units and about 1,500 colony forming units, about 10 colony forming units and 1,250 colony forming units, about 800 colony forming units and about 1,200 colony forming units, about 750 colony forming units and about 1,500 colony forming units, about 750 colony forming units and 1,250 colony forming units, or about 750 colony forming units and about 1,000 colony forming units.) In some examples, the viable microorganisms stabilize within a desired lower limit and a desired upper limit of colony forming units of between about 10³ colony forming units and about 10¹⁰ colony forming units, about 10³ colony forming units and about 10⁹ colony forming units, about 10³ colony forming units and about 10⁸ colony forming units, about 10³ colony forming units and about 10⁷ colony forming units, about 10³ colony forming units and about 10⁶ colony forming units, about 10³ colony forming units and about 10⁵ colony forming units, about 10³ colony forming units and about 10⁴ colony forming units, about 10⁵ colony forming units and about 10⁷ colony forming units, about 10⁷ colony forming units and about 10⁹ colony forming units, or about 10² colony forming units and about 10³ colony forming units. In other examples, the viable microorganisms stabilize within a desired lower limit and a desired upper limit of colony forming units of between about 20 colony forming units and about 150 colony forming units. In other examples, the viable microorganisms stabilize within a desired lower limit and a desired upper limit of colony forming units of between about 400 colony forming units and about 1,000 colony forming units. In other examples, the viable microorganisms stabilize within a desired lower limit and a desired upper limit of colony forming units of between about 0.5×10⁸ colony forming units and about 2.0×10⁸ colony forming units. In other examples, the viable microorganisms stabilize within a desired lower limit and a desired upper limit of colony forming units of between about 5,000 colony forming units and about 15,000 colony forming units.

In some cases, the desired upper limit and desired lower limit for the quantity of stabilized viable microorganisms are each selected from the following group: about 10¹⁰ colony forming units, about 10⁹ colony forming units, about 10⁸ colony forming units, about 10⁷ colony forming units, about 10⁶ colony forming units, about 100,000 colony forming units, about 90,000 colony forming units, about 80,000 colony forming units, about 70,000 colony forming units, about 60,000 colony forming units, about 50,000 colony forming units, about 40,000 colony forming units, about 30,000 colony forming units, about 20,000 colony forming units, about 10,000 colony forming units, about 9,000 colony forming units, about 8,000 colony forming units, about 7,000 colony forming units, about 6,000 colony forming units, about 5,000 colony forming units, about 4,000 colony forming units, about 3,000 colony forming units, about 2,000 colony forming units, about 1,000 colony forming units, about 900 colony forming units, about 800 colony forming units, about 700 colony forming units, about 600 colony forming units, about 500 colony forming units, about 400 colony forming units, about 300 colony forming units, about 200 colony forming units, about 100 colony forming units, about 90 colony forming units, about 80 colony forming units, about 70 colony forming units, about 60 colony forming units, about 50 colony forming units, about 40 colony forming units, about 30 colony forming units, about 20 colony forming units, about 10 colony forming units, about 5 colony forming units, and about 1 colony forming unit. Additional desired upper limit and desired lower limit quantity values of stabilized viable microorganisms included in any of the inoculating systems provided herein are contemplated, including all values within the ranges disclosed herein.

In some cases, any of the inoculating systems and devices provided herein can be manufactured in a batch. A batch refers to a group of products from the same production run made under the same manufacturing conditions from the same materials. In some cases, a batch may be manufactured that includes at least 3 inoculating systems (e.g., at least 10 inoculating systems, at least 100 inoculating systems, at least 1,000 inoculating systems, at least 10,000 inoculating systems, at least 100,000 inoculating systems, or greater than 1,000,000 inoculating systems). The mean quantity of viable microorganisms per device can be calculated for a batch of inoculation devices. In any of the inoculating systems and devices provided herein, the quantity of viable microorganisms may be within a standard deviation of about 50% or less, about 45% or less, about 40% or less, about 35% or less, about 30% or less, about 25% or less, about 20% or less. In some cases, the quantity of viable microorganisms stabilized on the transfer region of an inoculating system is preferably within a standard deviation of about 20% or less (e.g., about 15% or less, about 10% or less, about 5% or less, or about 2% or less).

In some cases, the quantity of viable microorganisms stabilized on the transfer region may be within a factor of 2 of the batch's mean quantity. In some cases, the quantity of viable microorganisms stabilized on the transfer region of an inoculation device (e.g., any of the inoculation devices provided herein) may be between about 50% to about 200% of the batch's mean quantity of viable microorganisms per device.

In some cases, the surface of the transfer region may have a surface wettability property. When the coating solution is deposited onto the surface of the transfer region, it exhibits a certain contact angle. The contact angle of the coating solution can be any value between about 40 degrees to about 120 degrees. In some cases, the contact angle of the coating solution can be between about 90 degrees to about 110 degrees.

In some cases, the transfer region may be rough or non-smooth. In some instances, the transfer region may have a surface roughness. The surface roughness of the transfer region can improve the viability, durability, and stability of the microorganisms. In some cases, the surface roughness may be a value between about 0.010 Ra microns to about 18.000 Ra microns. In some cases, the surface roughness may be about 0.60 Ra microns to about 2.80 Ra microns. In some cases, the surface roughness may be about 0.80 Ra microns to about 1.20 Ra microns. In some cases, the surface roughness may be about 0.80 Ra microns to about 1.00 Ra microns. In some cases, the surface roughness may be about 1.00 Ra microns to about 2.80 Ra microns.

In some cases, an additive may improve survivability of the viable microorganisms by improving adherence of the coating solution to the transfer region. In some cases, an additive may improve survivability of the viable microorganisms by reducing, minimizing, or eliminating detachment of the coating solution and viable microorganisms during or after the preservation process.

In some cases, the transfer region can be separated from a portion of the inoculation device that includes the handle region. In some cases, the transfer region can snap off from a portion of the inoculation device that includes the handle region. In some cases, the inoculation device may include a perforated section that is configured to enable the transfer region to be separated from the handle region of the inoculation device.

In some examples of any of the systems, devices, or methods provided herein, the viable microorganisms define an inoculum. In some examples, the systems, devices, or methods provided herein are intended to rapidly transfer viable microorganisms to a target (e.g., any of the targets described herein) from the inoculating member in a dry state without the need for an additional step to prepare the inoculum. Any of the inoculating systems provided herein can also be used to create a secondary inoculum (e.g., a liquid solution comprising known viable microorganisms that is further used to inoculate another target (e.g., agar media)). Described herein are apparatuses for storing viable microorganisms and rapidly creating a secondary inoculum. In some cases, the apparatuses provided herein are useful for storing viable microorganisms and rapidly creating a liquid secondary inoculum.

In some cases, any of the inoculating systems provided herein can be sealed and stored in a container (e.g., a moisture barrier container) for a storage time at a storage temperature (e.g., a desirable storage temperature range with a desired upper limit and a desired lower limit) prior to use in inoculating a target. In some cases, an inoculating system is sealed and stored in a container for at least about 1 day, at least about 2 days, at least about 3 days, at least about 5 days, at least about 7 days, at least about 14 days, at least about 21 days, at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 365 days, at least about 400 days, at least about 450 days, at least about 500 days, at least about 550 days, at least about 600 days, at least about 650 days, at least about 700 days, or at least about 750 days. Other storage times are contemplated.

Any of the inoculating systems provided herein can be sealed and stored in a container for a storage time (e.g., any of the storage times provided herein) at a temperature between about 2° C. and about 25° C. In some cases, an inoculating system may be stored at a temperature between about 2° C. and about 8° C. In some cases, an inoculating system may be stored at a temperature between about 18° C. and about 25° C. In some cases, an inoculating system may be stored at a temperature between about 20° C. and about 25° C. In some cases, an inoculating system may be stored at a temperature between about-26° C. and about 0° C. The desired upper limit and desired lower limit for a storage temperature may be any of the exemplary storage temperatures provided herein. Other storage temperatures (e.g., desired storage temperatures with an upper limit and a lower limit each between −26° C. and about 25° C.) are contemplated.

Provided herein are methods useful for storing viable microorganisms and rapidly transferring said viable microorganisms to a target. Methods are provided for rapidly transferring viable microorganisms to a target from an inoculating system (e.g., any of the inoculating systems described herein) in a dry state without the need for an additional step to prepare the inoculum. Methods for rapidly transferring viable microorganisms to a target at room temperature or ambient temperature are provided herein. In some cases, the viable microorganisms inoculate a target in 60 seconds or less (e.g., 50 seconds or less, 40 seconds or less, 30 seconds or less, 20 seconds or less, 10 seconds or less, or 5 seconds or less). In some examples, the viable microorganisms are substantially transferred in 60 seconds or less (e.g., 50 seconds or less, 40 seconds or less, 30 seconds or less, 20 seconds or less, 10 seconds or less, or 5 seconds or less).

Any of the methods provided herein may be used to rapidly inoculate a biologically acceptable target, e.g., solid media, a liquid solution, or a microbial collection device. Disclosed herein are methods of storing a known inoculum and rapidly transferring the known inoculum to a biologically acceptable target. In some examples of the systems and methods herein, the viable microorganisms are transferred to the target at ambient temperature. In some cases, the target does not need to be warmed to an elevated temperature. In some cases of the systems and methods herein, the viable microorganisms may be transferred to the target at temperature greater than ambient temperature. In some examples, the inoculating system does not need to be equilibrated to room temperature or ambient temperature before transferring the inoculum. In other examples, the inoculating system is equilibrated to room temperature or ambient temperature before transferring the inoculum.

In some cases, the inoculum is capable of being transferred to a target directly from a dry state and there are no additional steps needed to prepare the inoculum. In some cases, the method includes simply removing the cover from the cover receiving region and contacting the transfer region of the inoculating member with the target. In any of the methods described herein, an inoculating system (e.g., any of the inoculating systems disclosed herein) may be stored until use at a temperature with a lower limit and an upper limit.

In some cases, the viable microorganisms are transferred to a solid media. In some cases, the solid media is agar media or dry sheet media. In some case, the target is a solid growth media and the viable microorganisms are substantially transferred to the solid growth media.

In some cases, and in the methods disclosed herein for rapidly inoculating a liquid target (e.g., a liquid solution) with an inoculum, the inoculation device transfers the viable microorganisms to the liquid target by contacting the liquid solution and moving, stirring, agitating, or maneuvering the inoculation device to cause, contribute, or accelerate the transfer of the inoculum to the target. In other cases, the inoculation device transfers the viable microorganisms to the liquid target by contacting the liquid solution without the need for moving, stirring, agitating, or maneuvering the inoculation device. In some cases, no additional equipment is needed to transfer the inoculum from the inoculation device to the target. In some cases, the inoculation device can provide sufficient mechanical action to release the viable microorganisms from the transfer region without the use of a separate instrument or device. In some cases, the viable microorganisms are transferred through mechanical action of the inoculation device.

In some cases, and in any of the inoculating systems, devices, or methods provided herein, the viable microorganisms and/or coating solution can be drop coated and dried onto a transfer region. The viable microorganisms may be drop coated and dried onto a transfer region without freezing the microorganisms, for example. In some cases, the viable microorganisms are dried by vacuum drying.

The microorganisms in any of the devices, apparatuses, inoculating systems, and methods provided herein can be a prokaryotic organism or a eukaryotic organism. Non-limiting examples of the types of microorganisms that can be included in any of the devices or methods provided herein include: bacteria, fungi, algae, protist, diatom, archaea, and cyanobacteria. In some embodiments, the microorganisms can comprise a single type of microorganism, at least two types of microorganisms, at least three types of microorganisms, at least four types of microorganisms, at least five types of microorganisms, or more than five types of microorganisms.

In some instances, the microorganisms can comprise a homogenous population of identical cells. In some embodiments, the microorganisms can comprise a heterogeneous population of microbial cells. In some embodiments, the microorganisms in any of the devices, apparatuses, inoculating systems, and methods provided herein can comprise one organism, at least two different organisms, at least three different organisms, at least four different organisms, at least five different organisms, at least six different organisms, at least seven different organisms, at least eight different organisms, at least nine different organisms, at least ten different organisms, at least twenty different organisms, at least thirty different organisms, or more than thirty different organisms.

In some embodiments, the microorganisms can comprise one species, at least two species, at least three species, at least four species, at least five species, at least six species, at least seven species, at least eight species, at least nine species, at least ten species, or more than ten species of microorganisms. In some embodiments, the microorganisms can comprise one strain, at least two strains, at least three strains, at least four strains, at least five strains, at least six strains, at least seven strains, at least eight strains, at least nine strains, at least ten strains, at least fifteen strains, at least twenty strains, at least twenty-five strains, at least thirty strains, at least thirty-five strains, at least forty strains, at least forty-five strains, at least fifty strains, or more than fifty strains of microorganisms. In some examples, the microorganisms include a gram-positive bacteria or a gram-negative bacteria. In some examples, the microorganisms include a fungi (e.g., a yeast). The microorganisms in any of the devices, apparatuses, inoculating systems, and methods provided herein can include a spore.

In some embodiments, the microorganism is selected from the group of: Acetobacter spp., Acetonema spp., Achromobacter spp., Acidithiobacillus spp., Acinetobacter spp., Actinobacillus spp., Actinomyces spp., Aerococcus spp., Aeromonas spp., Aggregatibacter spp., Agrobacterium spp., Alcaligenes spp., Alicyclobacillus spp., Alkalibacillus spp., Alternaria spp., Ammoniphilus spp., Amphibacillus spp. Anaerobacter spp., Anaerospora spp., Anaplasma spp., Aneurinibacillus spp., Anoxybacillus spp., Arcanobacterium spp., Arthrobacter spp., Aspergillus spp., Aureobasidium spp. Azorhizobium spp., Azotobacter spp., Bacillus spp., Bacteroides spp., Bartonella spp. Beggiatoa spp., Bifidobacterium spp., Brevibacterium spp., Brevibacillus spp., Brevundimonas spp., Bordetella spp., Borrelia spp., Brochothrix spp., Brucella spp., Burkholderia spp., Caldanaerobacter spp., Caloramator spp., Calymmatobacterium spp., Caminicella spp., Campylobacter spp., Candida spp., Capnocytophaga spp., Cedecea spp., Cellulosimicrobium spp., Cerasibacillus spp., Chaetomium spp., Chlamydia spp., Chlamydophila spp., Chryseobacterium spp., Citrobacter spp., Cladosporium spp., Clostridioides spp., Clostridium spp., Clostridiisalibacter spp., Cohnella spp., Corynebacterium spp., Coxiella spp., Cronobacter spp., Cryptococcus spp., Curtobacterium spp., Cutibacterium spp., Deinococcus spp., Dendrosporobacter spp., Desulfotomaculum spp., Desulfosporomusa spp., Desulfosporosinus spp., Desulfovibrio spp., Desulfovirgula spp., Desulfunispora spp., Desulfurispora spp., Edwardsiella spp., Eggerthella spp., Ehrlichia spp., Eikenella spp., Elizabethkingia spp., Enterobacter spp., Enterococcus spp., Erysipelothrix spp., Escherichia spp., Eurotium spp., Ferrobacillus spp., Filifactor spp., Filobacillus spp., Finegoldia spp., Fluoribacter spp., Francisella spp., Fusarium spp., Fusobacterium spp., Gallionella spp., Gardnerella spp., Gelria spp., Geobacillus spp., Geosporobacter spp., Geotrichum spp., Gracilibacillus spp., Haemophilus spp., Hafnia spp., Halobacillus spp., Halonatronum spp., Hanseniaspora spp., Helicobacter spp., Heliobacterium spp., Heliophilum spp., Herminiimonas spp., Hormoconis spp., Issatchenkia spp., Klebsiella spp., Kocuria spp., Laceyella spp., Lactobacillus spp., Lactococcus spp., Legionella spp., Lentibacillus spp., Leptothrix spp., Listeria spp., Lysinibacillus spp., Malassezia spp., Mannheimia spp., Mahella spp., Megasphaera spp., Metabacterium spp., Methanobacterium spp., Methylobacterium spp., Meyerozyma spp., Microbacterium spp., Micrococcus spp., Microsporum spp., Moorella spp., Moraxella spp., Morganella spp., Mucor spp., Mycobacterium spp., Mycoplasma spp., Myroides spp., Natroniella spp., Neisseria spp., Nocardia spp., Oceanobacillus spp., Ochrobactrum spp., Oligella spp., Orenia spp., Ornithinibacillus spp., Oxalophagus spp., Oxobacter spp., Paenibacillus spp., Parabacteroides spp., Paraliobacillus spp., Parvimonas spp., Pasteurella spp., Pediococcus spp., Pelospora spp., Pelotomaculum spp., Penicillium spp., Peptostreptococcus spp., Piscibacillus spp., Planifilum spp., Pluralibacter spp., Pneumocystis spp., Pontibacillus spp., Porphyromonas spp., Prevotella spp., Propionibacterium spp., Propionispora spp., Proteus spp., Prototheca spp., Providencia spp., Pseudomonas spp., Ralstonia spp., Raoultella spp., Rhizobium spp., Rhizopus spp., Rhodococcus spp., Rickettsia spp., Rochalimaea spp., Rothia spp., Saccharomyces spp., Salinibacillus spp., Salmonella spp., Salsuginibacillus spp., Seinonella spp., Serratia spp., Shewanella spp., Shigella spp., Shimazuella spp., Sinorhizobium spp., Sphingobacterium spp., Sphingomonas spp., Spirillum spp., Sporacetigenium spp., Sporidiobolus spp., Sporoanaerobacter spp., Sporobacter spp., Sporobacterium spp., Sporohalobacter spp., Sporolactobacillus spp., Sporomusa spp., Sporosarcina spp., Sporotalea spp., Sporotomaculum spp., Staphylococcus spp., Stenotrophomonas spp., Stomatococcus spp., Streptococcus spp., Streptomyces spp., Syntrophomonas spp., Syntrophospora spp., Talaromyces spp., Tenuibacillus spp., Tepidibacter spp., Terribacillus spp., Thalassobacillus spp., Thermoacetogenium spp., Thermoactinomyces spp., Thermoalkalibacillus spp., Thermoanaerobacter spp., Thermoanaerobacterium spp., Thermoanaeromonas spp., Thermobacillus spp., Thermoflavimicrobium spp., Thermovenabulum spp., Thiobacillus spp., Thiothrix spp., Treponema spp., Trichophyton spp., Trichosporon spp., Trueperella spp., Tuberibacillus spp., Ureaplasma spp., Veillonella spp., Vibrio spp., Virgibacillus spp., Viridans spp., Vulcanobacillus spp., Wallemia spp., Wolbachia spp., Yarrowia spp., Yersinia spp., and Zygosaccharomyces spp.

In some embodiments, the microorganism is selected from the group of: Acetobacter aurantius, Acidithiobacillus thiooxidans, Acinetobacter baumannii (e.g., ATCC® 19606™), Actinomyces israelii, Agrobacterium radiobacter, Agrobacterium tumefaciens, Anaplasma phagocytophilum, Arthrobacter chlorophenolicus, Arthrobacter crystallopoietes, Arthrobacter luteus, Aspergillus brasiliensis (e.g., ATCC® 16404™), Aspergillus flavus (e.g., ATCC® 9643™), Aspergillus fumigatus (e.g., ATCC® 204305™), Aspergillus niger (e.g., ATCC® 6275™), Aspergillus terreus (e.g., ATCC® 1012™), Aureobasidium pullulans, Azorhizobium caulinodans, Azotobacter vinelandii, Bacillus anthracis, Bacillus atrophaeus, Bacillus brevis, Bacillus cereus, Bacillus fusiformis, Bacillus licheniformis, Bacillus megaterium, Bacillus mycoides, Bacillus stearothermophilus, Bacillus subtilis (e.g., ATCC® 6633™), Bacillus thuringiensis, Bacteroides fragilis, Bacteroides gingivalis, Bartonella henselae, Bartonella quintana, Beggiatoa alba, Bordetella bronchiseptica, Bordetella pertussis (e.g., ATCC® 12743™), Borrelia burgdorferi, Brevundimonas diminuta (e.g., ATCC® 19146™), Brucella abortus, Brucella melitensis, Brucella suis, Burkholderia mallei, Burkholderia pseudomallei, Burkholderia cepacia (e.g., ATCC® 25416™), Calymmatobacterium granulomatis, Campylobacter coli, Campylobacter fetus, Campylobacter jejuni (e.g., ATCC® 33291™ and ATCC® 29428™), Campylobacter pylori, Candida albicans (e.g., ATCC® 2091™ and ATCC® 10231™), Candida auris, Candida dubliniensis, Candida krusei, Candida glabrata, Candida lusitaniae, Candida parapsilosis, Candida tropicalis, Chlamydia trachomatis, Chlamydophila pneumoniae, Chlamydophila psittaci, Cladosporium cladosporioides, Cladosporium resinae, Clostridium botulinum, Clostridium difficile (e.g., ATCC® 43598™), Clostridium perfringens, Clostridium sporogenes (e.g., ATCC® 11437™ and ATCC® 19404™), Clostridium tetani, Corynebacterium ammoniagenes, Corynebacterium diphtheriae, Corynebacterium fusiforme, Corynebacterium glutamicum, Corynebacterium stationis, Coxiella burnetii, Cronobacter sakazakii (e.g., ATCC® 12868™), Cryptococcus neoformans, Desulfovibrio africanus, Desulfovibrio desulfuricans, Desulfovibrio salixigens, Desulfovibrio vulgaris, Desulfotomaculum orientis, Desulfotomaculum nigrificans, Ehrlichia chaffeensis, Enterobacter aerogenes (e.g., ATCC® 13048™), Enterobacter cloacae, Enterococcus avium, Enterococcus durans, Enterococcus faecalis (e.g., ATCC® 29212™ and ATCC® 51575™), Enterococcus faecium (e.g., ATCC® 35667™ and ATCC® 51559™), Enterococcus gallinarum, Enterococcus hirae, Enterococcus maloratus, Escherichia coli (e.g., ATCC® 8739™, ATCC® 25922™, ATCC® 10536™, ATCC® 11229™, and ATCC® 35150™), Ferrobacillus ferrooxidans, Fluoribacter bozemanae, Francisella tularensis, Fusobacterium nucleatum, Gallionella ferruginea, Gardnerella vaginalis, Geobacillus stearothermophilus, Haemophilus ducreyi, Haemophilus influenzae (e.g., ATCC® 10211™), Haemophilus parainfluenzae, Haemophilus pertussis, Haemophilus vaginalis, Helicobacter pylori, Hormoconis resinae, Klebsiella oxytoca (e.g., ATCC® 13182™), Klebsiella pneumoniae (e.g., ATCC® 4352™ and ATCC® 51503™), Kocuria rhizophila (e.g., ATCC® 9341™), Lactobacillus acidophilus, Lactobacillus bulgaricus, Lactobacillus casei, Lactobacillus plantarum, Lactococcus lactis, Legionella pneumophila (e.g., ATCC® 33153™), Leptothrix ochracea, Leptothrix discophora, Leptothrix cholodnii, Leptothrix lopholea, Leptothrix mobilis, Listeria monocytogenes (e.g., ATCC® 19117™, ATCC® 19111™, and ATCC® 7644™), Methanobacterium extroquens, Microbacterium multiforme, Micrococcus luteus, Moraxella catarrhalis, Moraxella osloensis, Mycobacterium avium, Mycobacterium bovis (e.g., ATCC® 35743™), Mycobacterium diphtheriae, Mycobacterium intracellulare, Mycobacterium leprae, Mycobacterium lepraemurium, Mycobacterium phlei, Mycobacterium smegmatis, Mycobacterium terrae (e.g., ATCC® 15755™), Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Paenibacillus glucanolyticus, Pasteurella multocida, Pasteurella tularensis, Penicillium chrysogenum, Pluralibacter gergoviae, Pneumocystis carinii, Pneumocystis murina, Porphyromonas gingivalis, Prevotella melaninogenica, Proteus mirabilis (e.g., ATCC® 9240™), Proteus vulgaris, Pseudomonas aeruginosa (e.g., ATCC® 9027™ and ATCC® 15442™), Pseudomonas fluorescens, Pseudomonas putida, Ralstonia pickettii, Rhizobium leguminosarum, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella bongori, Salmonella carrau, Salmonella concord, Salmonella enterica (e.g., ATCC® 10708™, ATCC® 6539™, ATCC® 4931™, ATCC® 13311™, and ATCC® 14028™), Salmonella enteritidis, Salmonella infantis, Salmonella newport, Salmonella schwarzengrund, Salmonella typhi, Salmonella typhimurium, Serratia marcescens (e.g., ATCC® 14756™ and ATCC® 8100™), Shewanella oneidensis, Shewanella putrefaciens, Shigella dysenteriae (e.g., ATCC® 11835™), Shigella flexneri (e.g., ATCC® 29508™), Shigella sonnei (e.g., ATCC® 11060™ and ATCC® 25931™), Sinorhizobium meliloti, Spirillum volutans, Staphylococcus aureus (e.g., ATCC® 6538™, ATCC® 29737™, ATCC® BAA-1683™, and ATCC® 33592™), Staphylococcus epidermidis (e.g., ATCC® 12228™), Staphylococcus hominis, Staphylococcus saprophyticus, Stenotrophomonas maltophilia, Stomatococcus mucilaginous, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus gordonii, Streptococcus lactis, Streptococcus mitior, Streptococcus mitis, Streptococcus mutans, Streptococcus oralis, Streptococcus pneumoniae (e.g., ATCC® 6305™), Streptococcus pyogenes (e.g., ATCC® 12384™ and ATCC® 19615™), Streptococcus rattus, Streptococcus salivarius, Streptococcus sanguis, Streptococcus sobrinus, Thiobacillus concretivorus, Thiobacillus thioparus, Treponema pallidum, Treponema denticola, Trichophyton interdigitale (e.g., ATCC® 9533™), Trichophyton mentagrophytes (e.g., ATCC® 9533™), Trichosporon asahii, Vibrio cholerae (e.g., ATCC® 11623™), Vibrio comma, Vibrio parahaemolyticus, Vibrio vulnificus, Viridans streptococci, Yersinia enterocolitica (e.g., ATCC® 23715™), Yersinia pestis, and Yersinia pseudotuberculosis, including drug-resistant strains thereof. Additional microorganisms that can be included in any of the devices or methods provided herein are contemplated.

The microorganisms may be naturally occurring or genetically modified. In some instances, the microorganism may be genetically modified, e.g., to exhibit fluorescence under certain conditions. The microorganism may be sourced from a laboratory collection or an environmental sample. In some examples, the microorganism is sourced from a preserved collection of reference microorganisms (e.g., ATCC®, NCTC, NCIMB, WDCM, CECT, NCPF, DSM, NBRC, ACM, or IMVS). In other instances, the microorganism is isolated from an environmental sample, e.g., from a human subject or from a surface in a manufacturing facility. Other sources and other methods of collecting and isolating microorganisms are known in the art and may be utilized.

A stabilizing mixture comprising stabilizing agents can be added to the known quantity/concentration of microorganisms. The stabilizing agents can be included to improve the stability of the microorganisms through the coating/drying process and over time in storage. In some embodiments, an additive may be included to improve other properties of the device and its use, e.g., the transfer efficiency of the microorganisms from the device to a target. Any of the exemplary stabilizing agents provided herein may be included as an additive to modify properties other than stability. Additional additives that can be included to modify properties other than stability are also contemplated.

Non-limiting examples of stabilizing agents that can be included in any of the devices and methods provided herein include: a sugar (e.g., a monosaccharide, a disaccharide, a reducing sugar, or a non-reducing sugar), a polyol, a polymer (e.g., an oligosaccharide, a polysaccharide, a cellulose-derivative, or a synthetic polymer), an antioxidant, an amino acid, a surfactant, and a buffer.

Non-limiting examples of sugars that can be included in any of the devices and methods provided herein include: glucose, fructose, xylose, arabinose, sorbose, mannose, rhamnose, galactose, trehalose, maltose, lactose, sucrose, melibiose, maltulose, iso-maltulose, and lactulose.

Non-limiting examples of polymers that can be included in any of the devices and methods provided herein include: raffinose, stachyose, melezitose, mannotriose, maltodextrin, dextran, starch, inulin, ficoll, alginate, chitosan, methylcellulose, hydroxypropyl cellulose, hydroxyethyl cellulose, hypromellose, xanthan gum, guar gum, pectin, carrageen, galactomannan, gellan gum, cellulose acetate phthalate, carboxy-methyl-cellulose, a salt of alginic acid (e.g., sodium alginate), hydroxyl propyl methyl cellulose, gum acacia, locust bean gum, hydroxyethyl cellulose, hydroxypropyl methylcellulose, polyvinyl alcohol, polyvinyl pyrrolidone, gelatin (e.g., hydrolyzed gelatin and unhydrolyzed gelatin), and polyglycolic acid.

Non-limiting examples of polyols that can be included in any of the devices and methods provided herein include: sorbitol, arabitol, xylitol, mannitol, erythritol, threitol, and glycerol.

Non-limiting examples of antioxidants that can be included in any of the devices and methods provided herein include: ascorbic acid, citric acid, acetic acid, a tocopherol, propyl gallate, tertiary butylhydroquinone, butylated hydroxyanisole, and butylated hydroxytoluene.

Non-limiting examples of amino acids that can be included in any of the devices and methods provided herein include: glycine betaine, sodium glutamate, cysteine, cystine, histidine, and methionine.

Non-limiting examples of buffers that can be included in any of the devices and methods provided herein include: a potassium phosphate (e.g., monopotassium phosphate), a sodium phosphate (e.g., monosodium phosphate and disodium phosphate), sodium acetate, sodium citrate, sodium succinate, histidine, imidazole, ammonium bicarbonate, a carbonate, [Tris(hydroxymethyl)methylamino]propanesulfonic acid (TAPS), 2-(Bis(2-hydroxyethyl)amino) acetic acid (Bicine), Tris(hydroxymethyl)aminomethane (Tris), 3-[N-Tris(hydroxymethyl)methylamino]-2-hydroxypropanesulfonic acid (Tricine), 3-[N-Tris(hydroxymethyl)methylamino]-2-hydroxypropanesulfonic acid (TAPSO), 4-(2-hydroxyethyl)-1-piperazineethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), Piperazine-N,N′-bis(2-ethanesulfonic acid) (PIPES), Dimethylarsenic acid (Cacodylate), 2-(N-morpholino) ethanesulfonic acid (MES), and N-cyclohexyl-2-aminoethanesulfonic acid (CHES).

Non-limiting examples of surfactants that can be included in any of the devices and methods provided herein include: a polysorbate (e.g., polysorbate 20, polysorbate 40, polysorbate 60, or polysorbate 80), a poloxamer (e.g., PLURONICS™), Triton X-100, polyethylene glycol, polypropylene glycol, polyethylene glycol/polypropylene glycol block copolymers, polyethylene glycol alkyl ethers, polypropylene glycol alkyl ethers, and polyethylene glycol/polypropylene glycol ether block copolymers.

Non-limiting examples of other stabilizing agents that can be included in any of the devices and methods provided herein include: milk (e.g., skimmed milk), monosodium glutamate, liquid growth medium, and propylene glycol. Other stabilizing agents that can be included in any of the devices or methods provided herein are contemplated.

In some instances, the stabilizing mixture may include a sugar. For example, the stabilizing mixture may include a non-reducing sugar such as sucrose. In some instances, the stabilizing mixture may include about 0.1% to about 15% of a stabilizing agent, for example, a sugar or a polyol (e.g., about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%). In some of these and in other instances, the stabilizing mixture may include an antioxidant. In some instances, the stabilizing mixture may include about 0.01% to about 10% of an antioxidant (e.g., about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, or about 0.1% to about 0.5%). In some of these and in other instances, the stabilizing mixture may include ascorbic acid. In some of these and in other instances, the stabilizing mixture may include a surfactant. In some instances, the stabilizing mixture may include about 0.001% to about 2% of a surfactant (e.g., about 0.001% to about 1.9%, about 0.001% to about 1.8%, about 0.001% to about 1.7%, about 0.001% to about 1.6%, about 0.001% to about 1.5%, about 0.001% to about 1.4%, about 0.001% to about 1.3%, about 0.001% to about 1.2%, about 0.001% to about 1.1%, about 0.001% to about 1.0%, about 0.001% to about 0.9%, about 0.001% to about 0.8%, or about 0.001% to about 0.7%, about 0.001% to about 0.6%, about 0.001% to about 0.5%, about 0.001% to about 0.4%, about 0.001% to about 0.3%, about 0.001% to about 0.2%, about 0.001% to about 0.1%, or about 0.001% to about 0.01%). For example, the stabilizing mixture may include a nonionic surfactant such as Triton X-100. In some of these and in other instances, the stabilizing mixture may include an amino acid. In some of these and in other instances, the stabilizing mixture may include a protein. In some of these and in other instances, the stabilizing mixture may include a salt. In some of these and in other instances, the stabilizing mixture may include a polymer. In some of these and in other instances, the stabilizing mixture may include a buffer. For example, the stabilizing mixture may include a phosphate buffer such as phosphate buffered saline. In some of these and in other instances, the stabilizing mixture may include tris(hydroxymethyl)aminomethane. In some of these and in other instances, the stabilizing mixture may include 2-[4-(2,4,4-trimethylpentan-2-yl) phenoxy]ethanol. In some of these and in other instances, the stabilizing mixture may include a growth nutrient. In some of these and in other instances, the stabilizing mixture may include a broth. In one embodiment, the stabilizing mixture includes 8-12% of a sugar (e.g., a non-reducing sugar, e.g., sucrose), 5-7% of an antioxidant (e.g., ascorbic acid), and 0.01-0.1% of a surfactant (e.g., Triton X-100). In a preferred embodiment, the stabilizing mixture includes 10% of a sugar (e.g., a non-reducing sugar, e.g., sucrose), 6% of an antioxidant (e.g., ascorbic acid), and 0.04% of a surfactant (e.g., Triton X-100). In another embodiment, the stabilizing mixture includes between 12-15% of a sugar (e.g., a non-reducing sugar, e.g., sucrose), 5-10% of an antioxidant (e.g., ascorbic acid), and optionally 0.01-0.1% of a surfactant (e.g., Triton X-100).

A suitable volume of the stabilizing mixture mixed with the known quantity/concentration of microorganisms (e.g., which may be referred to as the coating solution or mixture) may be disposed on the transfer region 14. For example, 1-10 microliters of the coating solution may be disposed onto the transfer region 14. This may include pipetting the coating solution onto the transfer region 14.

In some embodiments of any of the devices, apparatuses, and inoculating systems provided herein, a volume of between 1 to 200 microliters of the coating solution may be applied onto the transfer region 14. In some embodiments, 5 microliters or less (e.g., 4 microliters or less, 3 microliters or less, 2 microliters or less, 1 microliters or less, 900 nanoliters or less, 800 nanoliters or less, 700 nanoliters or less, 600 nanoliters or less, 500 nanoliters or less, 400 nanoliters or less, 300 nanoliters or less, 200 nanoliters or less, 100 nanoliters or less, 50 nanoliters or less, 25 nanoliters or less, 10 nanoliters or less, 1 nanoliter or less, 900 picoliters or less, 800 picoliters or less, 700 picoliters or less, 600 picoliters or less, 500 picoliters or less, 400 picoliters or less, 300 picoliters or less, 200 picoliters or less, 100 picoliters or less, 50 picoliters or less, 25 picoliters or less, or 10 picoliters or less) of the coating solution is applied onto the transfer region. In some instances, the volume of coating solution may be applied to the transfer region in a single application or by multiple applications.

In some instances, a spreading device may be used to spread the coating solution along the transfer region 14. However, spreading is not required. The coating solution may be allowed to air dry (e.g., dry at room temperature) for a suitable time period such as 1-60 minutes. In some embodiments of any of the devices, apparatuses, and inoculating systems provided herein, the coating solution may be allowed to air dry for 1-50 minutes, 1-45 minutes, 1-40 minutes, 1-35 minutes, 1-30 minutes, 1-25 minutes, 1-20 minutes, 1-15 minutes, 1-10 minutes, 1-5 minutes, or 1 minute or less. In some embodiments of any of the devices, apparatuses, and inoculating systems provided herein, the coating solution may be allowed to air dry at room temperature. In other embodiments, the coating solution may be allowed to air dry at an elevated temperature (e.g., between 25° C. to 40° C.).

In some embodiments of any of the devices, apparatuses, and inoculating systems provided herein, the quantity of microorganisms coated onto the transfer region can be 1-100 CFU, 1-1,000 CFU, or greater than 1,000 CFU. In other embodiments of any of the devices apparatuses, and inoculating systems provided herein, the quantity of viable microorganisms coated onto the transfer region is not known and/or is not pre-determined. In other embodiments, the quantity of viable microorganisms coated onto the transfer region comprises at least one viable microorganism. In some embodiments of any of the devices, apparatuses, and inoculating systems provided herein, no microorganisms are coated onto the transfer region of the device or onto any other part of the device, and the device is used to spread microorganisms that have otherwise been deposited onto a target.

In some examples after drying the inoculating system can comprise between about 0% to about 10% (e.g., between 0% to 5%, between 0% to 2.5%, or between 0% to 1%) of residual water content or residual moisture content by weight (e.g., w/w). In some examples, the pre-determined quantity of microorganisms has a viability of 25% or more after 30 days (e.g., 30% of more after 30 days, 40% of more after 30 days, 50% of more after 30 days, 60% of more after 30 days, 70% of more after 30 days, 80% of more after 30 days, 90% of more after 30 days, or 100% of more after 30 days).

In some embodiments of any of the devices, apparatuses, and inoculating systems provided herein, the transfer region and/or the projection(s) along the transfer region is convex and is curved or rounded outward like the exterior of a sphere or circle.

The transfer of microorganisms to a target from any of the devices, apparatuses, and/or inoculating systems provided herein can be described in part by microbial transfer efficiency or efficacy. It can be appreciated that some microorganisms may be lost while coating onto the transfer region and/or during drying. For the purposes of this disclosure, the transfer efficiency or efficacy may be understood to be the percentage of viable microorganisms disposed on the transfer region that are transferred to the target during an inoculation. In other words, the transfer efficiency or efficacy is the percentage of dried viable microorganisms that are successfully transferred from the transfer region to the target during an inoculation. In some embodiments, the microbial transfer efficiency may be about 50% or more, or about 55% or more, or about 60% or more, or about 65% or more, or about 70% or more, or about 75% or more, or about 80% or more, or about 85% or more, or about 90% or more, or about 95% or more, or about 99% or more, or about or nearly 100%.

### EXAMPLES

The disclosure may be further clarified by reference to the following Examples, some of which are prophetic in nature, and serve to exemplify some embodiments, and not to limit the disclosure in any way.

### Example 1—Device for Direct-Transfer of Bacterial Cells to Agar Media

Staphylococcus aureus (ATCC® 6538™) was grown in nutrient broth for 48 hours at 35° C. The culture was then diluted 5.0E-4 into the coating solution. The coating solution contained synthetic broth (HiMedia M334-500G), sucrose (1% w/v, Aldon Corporation IS28231), and Triton X-100 (0.04% v/v, Electron Microscopy Sciences 22140) in DI water. The coating solution was prepared by first adding synthetic broth powder to DI water (16.9 g/L) followed by steam sterilization of the media. Sucrose was then added to the synthetic broth and dissolved to yield 1% w/v. Then Triton X-100 was added to the solution to yield 0.04% Triton X-100. This solution was then filter sterilized and the S. aureus cells were diluted into it in order to target <100 CFU/10 μL volume.

The devices were prepared by cutting circles (1.2″ diameter) of polyester film (PET) with adhesive backing (McMaster-Carr 8689K44), pealing the release liner from the back of the PET film and adhering the film to a polypropylene 50 ml conical tube cap (1.25″ diameter, 0.5″ tall). The assembled devices were then steam sterilized at 121° C. for 20 minutes.

The prepared coating solution was then dispensed (10 μL) onto the surface of the sterilized device and spread over the surface of the PET film with a sterile microbiology spreading rod. The coated devices were then dried at 36° C. for approximately 20 minutes or until the water in the coating solution was substantially evaporated. The devices may then be placed into a moisture barrier package such as an aluminum mylar pouch that also contains a desiccant and oxygen scavenger.

The assembled devices may then be removed from the packaging material. The devices (n=3) were then directly contacted to the surface of trypticase soy agar (TSA) media in a petri dish so that the coated surface of the device was in contact with the surface of the agar media. The device was contacted to the TSA surface for approximately 5 seconds to allow the S. aureus cells to transfer from the surface of the device to the surface of the agar. The devices were then disposed and the inoculated TSA petri dish was placed into a 35° C. incubator for approximately 18 hours. The colony counts are shown in Table 1.

TABLE 1
Colony Forming Units (CFUs) after direct transfer from device.

| Device # | CFU Number |
|---|---|
| 1 | 77 |
| 2 | 69 |
| 3 | 66 |

### Example 2—Device for Direct-Transfer of Arrayed Pattern of Bacterial Cells to Agar Media

Staphylococcus aureus (ATCC® 6538™) can be grown in nutrient broth for 48 hours at 35° C. The culture can be diluted 1.0E-4 into the coating solution. The coating solution may contain ascorbic acid (1% w/v), sucrose (2% w/v, Aldon Corporation IS28231), 1× phosphate buffered saline (PBS) in DI water. This solution can be sterilized and the S. aureus cells can be diluted into it in order to target between 1 and 20 cells per 100 nL volume.

The device can be thermoforming out of polypropylene plastic (40 mil, or 1 mm thick) into a device with dimensions 2.5″ diameter×1″ tall. The device can be sterilized by a number of know sterilization modalities.

The sterile device can then be coated with the prepared coating solution by dispensing 100 nL droplets onto the surface of the device in an array pattern (7×7 pattern for total of 49 drops) using an automated precision dispensing machine. The coated device is then allowed to air dry at room temperature until the H₂0 in the coating solution is substantially evaporated. The devices may then be placed into a moisture barrier package such as an aluminum mylar pouch or similar that also contains a desiccant and oxygen scavenger or a modified atmosphere.

The assembled device may be stored at 4° C., then removed from the packaging material at the point of use. The device can be directly contacted to the surface of trypticase soy agar (TSA) media in a petri dish so that the coated surface of the device is in contact with the surface of the agar media. The devices can be disposed and the inoculated TSA petri dish can be placed into a 35+/−2° C. incubator for approximately 18-24 hours. The CFUs can then be counted and are shown in Table 2.

TABLE 2
CFUs on TSA following contact with instant inoculator
device with arrayed coating pattern (n = 3).

| Device # | CFUs |
|---|---|
| 1 | 49 |
| 2 | 50 |
| 3 | 49 |

### Example 3—Device for Direct-Transfer of Bacterial Cells to Agar Media

Bacillus subtilis (ATCC® 6633™) was grown on trypticase soy agar (TSA) for 72 hours at 35° C. The spores were then harvested and purified. The coating solution was prepared by first adding synthetic broth powder to DI water (16.9 g/L) followed by steam sterilization of the media. Sucrose was then added to the synthetic broth and dissolved to yield 1% w/v. Then Triton X-100 was added to the solution to yield 0.04% Triton X-100. Finally, ascorbic acid was added to the solution to yield 0.1% w/v. This solution was then filter sterilized and the B. subtilis spores were diluted into it in order to target <100 CFU/100 μL volume.

The devices were injection molded out of polypropylene. Each device included 40 five mm diameter projections on the transfer region. The transfer region was about 2.25 inches in diameter and the handle region was about 1 inch tall. The devices were steam sterilized prior to coating.

The prepared coating solution was then dispensed (2.5 μL/projection) onto the surface of the sterilized device. The coated devices were then dried at room temperature for approximately 20 minutes or until the water in the coating solution was substantially evaporated. Sterile polypropylene caps were then placed over the transfer region of the coated devices. The devices (4 per pouch) were then placed into an aluminum mylar pouch that contained a desiccant and an oxygen scavenger. The pouch was then sealed with a heat sealer.

The sealed pouches were held at room temperature for approximately 1 hour in order to let the devices equilibrate to the package atmosphere. The pouch was then opened and a portion of the devices (n=2) were then removed from the packaging material. These devices were considered the ‘time zero’ pull point and were directly contacted to the surface of trypticase soy agar (TSA) media in a petri dish so that the coated surface of the device was in contact with the surface of the agar media. The device was contacted to the TSA surface for approximately 10 seconds, with a spreading motion, to allow B. subtilis to transfer from the surface of the device to the surface of the agar. The devices were then disposed and the inoculated TSA petri dish was placed into a 35° C. incubator for approximately 18 hours. The colony counts are shown in Table 3. The remaining devices in the pouch were immediately sealed and the package was placed at 35° C. for an accelerated aging study. After 2 days of storage at 35° C. (representative of 4-6 months at 4° C.) were removed from the package and directly contacted to the agar and incubated as was described for the time zero pull point. The CFU results are shown in Table 3.

TABLE 3
CFUs after direct transfer from device.

| Device # | T = 0 Pull | Pull 1 (following storage at 35 °C for 2 days) |
|---|---|---|
| 1 | 81 | 91 |
| 2 | 81 | 94 |

### Example 4—Device for Direct-Transfer of Bacterial Cells to a Sample Collection Sponge

Escherichia coli (ATCC® 8739™) was grown in synthetic broth for approximately 24 hours at 35° C. E. coli cells were diluted into a sterile coating solution containing sucrose (0.4% w/v), ascorbic acid (0.5%), casamino acids (0.6%), and triton X100 (0.04%) to yield approximately 5.0×10³ CFU/device.

Devices for direct-transfer of bacterial cells to a sample collection sponge were injection molded out of polypropylene. Each device included forty, five mm diameter projections on the transfer region. The transfer region was about 2.25 inches in diameter and the handle region was about 1 inch tall. The devices and caps for the device were steam sterilized prior to coating.

The coating solution containing the E. coli cells was then dispensed (2.5 μL/projection, total coating volume of 100 μL) onto the surface of the sterilized device. The coated devices were then vacuum dried for approximately 20 with a final pressure of approximately 500 mTorr. Sterile polypropylene caps were then placed over the transfer region of the coated devices. The devices were then placed into an aluminum mylar pouch that contained a desiccant and an oxygen scavenger. The pouch was then sealed with a heat sealer and stored at 2-8° C.

On the day of testing, the pouches were removed from 2-8° C. storage and placed at ambient conditions for 15 minutes. The coated devices were then removed from the pouches. The target in this study was World Bioproducts sponges (SAM-EZ-10NB-PUR) and 3M sponges (HS10NB). Both of these sponges come pre-wetted with neutralizing buffer from the respective manufacturer, and were directly contacted, in accordance with their IFU's, with the coated transfer region of the prepared positive control devices. The sponges were then placed into the plastic bags they came in and the neutralizing buffer was squeezed out of the sponges to recover the microbes. The recovery solution was then sampled and plated onto TSA. The TSA plates were incubated at 35° C. for 24 hours. The CFU results recovered from the sponges are shown in Table 4, the total volume of liquid recovered from each sponge was used to calculate the CFU/device. In addition, a control set of devices were sampled directed by submerging the transfer region in 10 mL of phosphate buffered saline in a sterile petri dish. A sample of this recovery buffer was then plated on TSA to determine the number of viable cells per device. Average viable CFU for the control devices was determined to be 4.9×10³ CFU/device. This number was used to determine the percentage recovery for each sponge type.

TABLE 4
CFUs after direct transfer from device.

| | |
|---|---|
| CFU/device | % Recovery |

| | WB | 3M | WB | 3M |
|---|---|---|---|---|
| 1 | 1.17E+03 | 2.42E+03 | 24% | 49% |
| 3 | 1.03E+03 | 1.78E+03 | 21% | 36% |
| 4 | 2.05E+03 | 1.33E+03 | 42% | 27% |
| 5 | 1.28E+03 | 1.04E+03 | 26% | 21% |
| 6 | 1.30E+03 | 1.78E+03 | 27% | 36% |
| Average | 1.37E+03 | 1.67E+03 | 28% | 34% |

### Example 5—Device for Direct-Transfer of Bacterial Cells to a Liquid Target

Staphylococcus aureus (ATCC® 6538™) can be grown in nutrient broth for 48 hours at 35° C. The culture can be diluted into the coating solution. The coating solution may contain synthetic broth (HiMedia M334-500G), sucrose (1% w/v, Aldon Corporation IS28231), and Triton X-100 (0.04% v/v, Electron Microscopy Sciences 22140) in DI water. This solution may be filter sterilized and the S. aureus cells can be diluted into the coating solution in order to target <100 CFU/5 μL volume.

Device for direct-transfer of bacterial cells to a liquid target were prepared by cutting 8 inch sections of polypropylene rod (¼″ outside diameter) and placing a 1.5″ section of silicone tubing at one end of the rod to create a handle region. Hollow polypropylene tubing (¼″ inside diameter) was then cut to approximately 7″ to be used as the cap or sleeve that protects the coated end of the rod. The assembled devices can then be steam sterilized at 121° C. for 20 minutes.

The prepared coating solution can then be dispensed (5 μL) onto the end of the rod opposite to the handle region. The coated devices can then be vacuum dried for approximately 20 minutes with a final pressure of approximately 500 mTorr. The devices can then be capped using a hollow tube to protect the coated transfer region. The assembled device may then be placed into a moisture barrier package such as an aluminum mylar pouch that also contains a desiccant and oxygen scavenger and stored at 2-8° C. until use.

The assembled devices may then be removed from the packaging material at the time of use. The devices can then be used to directly inoculate 10 mL of trypticase soy broth (TSB) by contacting the coated transfer region of the rod with the TSB and shaking the rod briefly to allow the viable cells to transfer to the broth. The devices can then be disposed into biohazardous waste stream and the inoculated TSB can be incubated at 35° C. for approximately 24 hours. The presence of turbidity in the TSB tubes can indicate that the TSB to be qualified can support low levels of microbial inoculums (<100 CFU). A negative control can be run as well to ensure that the TSB was not contaminated prior to inoculation. The expected results are shown in Table 5.

TABLE 5
Presence or absence of turbidity (+/−) in inoculated TSB tubes.

| Device # | Turbidity (+/−) |
|---|---|
| 1 | + |
| 2 | + |
| 3 | + |
| Negative | − |
| Control | |

### Example 6—Device for Direct-Transfer of Bacterial Cells to a Liquid Target for Quantitative Use

Pseudomonas aeruginosa (ATCC® 9027™) was grown in a nutrient broth at 35° C. A coating solution was prepared with case amino acids, sucrose, ascorbic acid and Triton X-100 in DI water. This solution was then sterilized and the P. aeruginosa cells were diluted into the coating solution in order to target approximately 500 CFU/device following drying.

Devices for direct-transfer of bacterial cells to a liquid sample tube were injection molded out of polypropylene. The devices were substantially in a stick/rod format with a length of 10.8 cm and a diameter of approximately 7 mm. The injection molded devices included a cap receiving region, a handle region, a transfer region, and vents. The transfer region was about 44 mm in length. The caps were also made of polypropylene and were 45 mm length×9 mm diameter. The devices and caps for the device were steam sterilized prior to coating and assembly.

The prepared coating solution was dispensed (2.5 μL) onto the end of the rod transfer region opposite to the handle region. The coated devices were then vacuum dried for approximately 20 minutes with a final pressure of approximately −29 inHg. The devices were then capped. The assembled devices were then placed into an aluminum mylar pouch that also contained 0.5 g silica desiccant and 20 cc oxygen scavenger. The pouches were then heat sealed and stored at 2-8° C. until use.

The assembled devices were then removed from the packaging material at the time of use. A sterile rehydration tube contained 1.1 mL of an appropriate buffer was prepared. The cap was then removed from the device and the transfer region was submerged in the rehydration solution in order to fully submerge the microbial coating on the end of the rod. The rod was swirled in the rehydration solution tube for <10 seconds. The microbial suspension in the rehydration solution tube was then briefly vortexed. 100 uL aliquots of the microbial suspension was then placed on onto trypticase soy agar for the purpose of qualifying the agar. The devices were then disposed into biohazardous waste stream and the inoculated TSA was incubated at 35° C. for approximately 24 hours. The TSA plates were then counted to determine both the CFU/100 uL injection and the CFU/device. The results are shown in Table 6.

TABLE 6
CFU results following rehydration of microbes on stick.

| | | | | |
|---|---|---|---|---|
| Days post | | | | |
| coating | rep | CFU/100 uL | CFU/stick | Avg |

| | | | | | |
|---|---|---|---|---|---|
| 7 Days | 1 | 54 | 59 | 622 | 567 |
| | 2 | 56 | 45 | 616 | |
| | 3 | 45 | 39 | 462 | |
| 41 Days | 1 | 47 | 53 | 550 | 486 |
| | 2 | 55 | 44 | 605 | |
| | 3 | 36 | 19 | 303 | |

### Example 7—Device for Direct-Transfer of Bacterial Cells to a Solid Target for Qualitative Use

Staphylococcus aureus (ATCC® 6538™) was grown in a nutrient broth at 35° C. A coating solution was prepared with ascorbic acid, sucrose, case amino acids and Triton X-100 in DI water. This solution was then sterilized and the S. aureus cells were diluted into the coating solution in order to target >1000 CFU/device following drying.

Devices for direct-transfer of bacterial cells to an agar media were injection molded out of polypropylene. The devices were substantially in a stick/rod format with a length of 10.8 cm and a diameter of approximately 7 mm. The injection devices included a cap receiving region, a handle region, a transfer region, and vents. The transfer region was about 44 mm in length. The caps were also made of polypropylene and were 45 mm length×9 mm diameter. The devices and caps for the device were steam sterilized prior to coating and assembly.

The prepared coating solution was dispensed (2.5 μL) onto the end of the rod transfer region opposite to the handle region. The coated devices were then vacuum dried for approximately 20 minutes with a final pressure of approximately −29 inHg. The devices were then capped. The assembled devices were then placed into an aluminum mylar pouch that also contained a 0.5 g silica desiccant and 20 cc oxygen scavenger. The pouches were then heat sealed and stored at 2-8° C. until use.

The assembled devices were then removed from the packaging material at the time of use. The cap was removed from the cap receiving region and the microbial coating on the rod contact directly with the agar media surface. The rod was then spread over the surface of the agar using a spread plating technique in order to obtain an isolated colony following incubation. The devices were then disposed into biohazardous waste stream and the inoculated TSA was incubated at 35° C. for approximately 24 hours. The TSA plates were then observed to determine if an isolated colony was obtained. The results are shown in Table 7.

TABLE 7

| Rep | Isolated colony (Yes/no) |
|---|---|
| 1 | Yes |
| 2 | Yes |
| 3 | Yes |

### Example 8—Device for Direct-Transfer of Bacterial Cells to a Sample Collection Swab

Escherichia coli (ATCC® 25922™) was grown in a nutrient broth at 35° C. A coating solution was prepared with case amino acids, sucrose, ascorbic acid, and Triton X-100 in DI water. This solution was then sterilized, and the E. coli cells were diluted into the coating solution in order to target approximately 4-5 log CFU/device following drying.

Devices for direct-transfer of bacterial cells to a swab sample collection device were injection molded out of polypropylene. The devices were substantially in a stick/rod format with a length of 10.8 cm and a diameter of approximately 7 mm. The injection molded devices included a cap receiving region, a handle region, a transfer region, and vents. The transfer region was about 44 mm in length. The caps were also made of polypropylene and were 45 mm length×9 mm diameter. The devices and caps for the device were steam sterilized prior to coating and assembly.

The prepared coating solution was dispensed (2.5 μL) onto the end of the rod transfer region opposite to the handle region. The coated devices were then vacuum dried for approximately 20 minutes with a final pressure of approximately −29 inHg. The devices were then capped. The assembled devices were then placed into an aluminum mylar pouch that also contained a 0.5 g silica desiccant and 20 cc oxygen scavenger. The pouches were then heat sealed and stored at 2-8° C. until use.

The assembled devices were then removed from the packaging material at the time of use. The cap was then removed from the device and the transfer region was contacted directly with a wetted swab sample collection devices. The swab was contacted with the tip of the transfer region for <10 seconds with circular swabbing motion. A sample of the swab sampling device was plated on TSA at time zero (TO) and the swabs were placed in a shipping container with ice and shipped overnight. Following 24 hours of shipping, the sample was plated on TSA to determine the CFU/swab. The results are shown in Table 8.

TABLE 8
Enverify Positive Control CFU results through swab shipping.

| | | CFU/plate | | | avg |
|---|---|---|---|---|---|
| Condition | rep | (10-2) | CFU/swab | log 10 | log10 |
| T0 | 1 | 263 | 2.89E+05 | 5.46 | 5.41 |
| | 2 | 204 | 2.24E+05 | 5.35 | |
| | 3 | 237 | 2.61E+05 | 5.42 | |
| Following 24 | 1 | 143 | 1.57E+05 | 5.20 | 5.11 |
| hrs of shipping | 2 | 90 | 9.90E+04 | 5.00 | |
| | 3 | 126 | 1.39E+05 | 5.14 | |

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1-191. (canceled)

192. An inoculation device comprising:
an elongate body comprising:
a cover receiving region encompassing a distal end of the elongate body;
a transfer region disposed within the cover receiving region of the elongate body; and
a handle region encompassing a proximal end of the elongate body;
a dried coating solution disposed on the transfer region of the elongate body, the dried coating solution comprising viable microorganisms and one or more additives that improve survivability of the viable microorganisms; and
a cover removably secured relative to the cover receiving region and configured to protect the viable microorganisms within the dried coating solution on the transfer region.

193. The inoculation device of claim 192, wherein the transfer region is disposed at the distal end of the elongate body.

194. The inoculation device of claim 193, wherein the cover has a cylindrical shape extending from a closed end of the cover to an open end of the cover, wherein, while the cover is secured to the elongate body, the closed end covers the transfer region without contacting the transfer region or the dried coating solution.

195. The inoculation device of claim 192, wherein the transfer region is configured to transfer the viable microorganisms to a target in a dry state without first rehydrating the dried coating solution.

196. The inoculation device of claim 195, wherein the transfer region is configured to transfer at least 90 percent of the viable microorganisms from the dried coating solution to the target in no more than 60 seconds at a temperature in a range from 15° C. to 25° C.

197. The inoculation device of claim 192, wherein the cover receiving region of the elongate body includes a securement region, and the cover is removably secured to the elongate body via an interference fit with the securement region.

198. The inoculation device of claim 192, wherein the elongate body has an overall length that is no less than 10 cm and no more than 30 cm.

199. The inoculation device of claim 192, wherein the cover receiving region of the elongate body has a length in a range from 10 percent to 60 percent of an overall length of the elongate body.

200. The inoculation device of claim 192, wherein the handle region of the elongate body has a length in a range from 40 percent to 90 percent of an overall length of the elongate body.

201. The inoculation device of claim 192, wherein the elongate body has a diameter that varies along a length of the elongate body and remains within a range from 0.5 cm to 1.5 cm.

202. The inoculation device of claim 192, further comprising a moisture barrier container, wherein the elongate body, the dried coating solution, and the cover are packaged within an interior of the moisture barrier container.

203. The inoculation device of claim 202, wherein the moisture barrier container contains a desiccant.

204. The inoculation device of claim 203, wherein the moisture barrier container also contains an oxygen scavenger.

205. The inoculation device of claim 202, wherein the moisture barrier container contains an inert gas.

206. The inoculation device of claim 192, wherein the elongate body is composed of a polymeric material.

207. An inoculation device comprising:
an elongate body comprising:
a cover receiving region encompassing a distal end of the elongate body;
a transfer region disposed at the distal end of the elongate body; and
a handle region encompassing a proximal end of the elongate body;
a dried coating solution disposed on the transfer region of the elongate body, the dried coating solution comprising viable microorganisms and one or more additives that improve survivability of the viable microorganisms; and
a cover removably secured relative to the cover receiving region and configured to protect the viable microorganisms within the dried coating solution on the transfer region,
wherein the transfer region is configured to transfer the viable microorganisms to a target in a dry state without first rehydrating the dried coating solution.

208. The inoculation device of claim 207, wherein the cover has a cylindrical shape extending from a closed end of the cover to an open end of the cover, wherein, while the cover is secured to the elongate body, the closed end covers the transfer region without contacting the transfer region or the dried coating solution.

209. The inoculation device of claim 207, wherein the elongate body has an overall length that is no less than 10 cm and no more than 30 cm.

210. The inoculation device of claim 207, wherein the cover receiving region of the elongate body includes a securement region, and the cover is removably secured to the elongate body via an interference fit with the securement region.

211. The inoculation device of claim 207, further comprising a moisture barrier container, wherein the elongate body, the dried coating solution, and the cover are packaged within an interior of the moisture barrier container.
